(19) United States
(12) Patent Application Publication
(10) Pub. No.: US 2017/0044222 A1
(43) Pub. Date: Feb. 16, 2017

(54) Anti-Microbial Coatings and Devices

(71) Applicant: Worcester Polytechnic Institute, Worcester, MA (US)

(72) Inventors: Todd E. Alexander, Worcester, MA (US); Lindsay D. Lozeau, Worcester, MA (US); Terri A. Camesano, Worcester, MA (US)

(21) Appl. No.: 15/235,778

(22) Filed: Aug. 12, 2016

Related U.S. Application Data
(60) Provisional application No. 62/205,206, filed on Aug. 14, 2015.

(51) Int. Cl. C07K 14/46; A61L 31/10; A61K 47/48; A61L 31/16

(52) U.S. Cl. CPC C07K 14/461; A61L 31/16; A61L 31/10; A61K 47/48215; A61K 38/00

(57) ABSTRACT

Compositions and methods for coating medical devices are provided. A coating composition may comprise a tether covalently attached to an anti-microbial peptide, the tether having sufficient length to permit the anti-microbial peptide to at least partially penetrate a membrane of a bacteria, upon contact of the anti-microbial peptide with the bacteria.

### RELATED APPLICATIONS

This application claims the benefit of and priority to U.S. Provisional Application Ser. No. 62/205,206, filed on Aug. 14, 2015, which is incorporated herein by reference in its entirety.

### STATEMENT OF GOVERNMENT SUPPORT

This invention was made with Government Support under Grant Numbers NSF DGE 1144804 and NSF-IIP 1439177 awarded by the National Science Foundation (NSF). The Government has certain rights in the invention.

### REFERENCE TO SEQUENCE LISTING

This specification includes a sequence listing provided on a compact disc, submitted herewith, which includes the file entitled 110697-013301 ST25.txt having the following size: 3,400 bytes which was created Aug. 12, 2016, the contents of which are incorporated by reference herein.

### FIELD

The disclosure relates generally to anti-microbial peptides and medical device coatings including such peptides.

### BACKGROUND

Increasing bacterial resistance due to the overuse of conventional antibiotics has led to an alarming rate of nosocomial infection and a desperate call for the development alternative antibiotics. The CDC estimates that these infections kill at least 23,000 people per year. Among the most promising alternatives are anti-microbial peptides (AMPs). AMPs are short peptide sequences (10-50 amino acids) and most are amphipathic and cationic (+2 to +9 charge). They are associated with the innate immunity of several species. Continuous exposure to a diverse array of pathogens requires AMPs to have broad spectrum activity against bacteria, fungi, viruses, and protozoa. AMPs are recognized for their potent activity against common gram negative and gram positive microbes, such as Escherichia coli (E. coli) and Staphylococcus aureus (S. aureus), which are common in hospitals and community environments. The development of anti-microbial resistance to current antibiotics has been identified as a serious threat.

AMPs use different mechanisms to kill bacteria than traditional antibiotics, making the development of bacterial resistance much less likely; however, these mechanisms differ between peptides and are not fully understood. Most proposed mechanisms involve physical AMP-membrane interactions, with AMPs using their positive charge and hydrophobicity to target anionic bacterial membranes, create pores, cause cell leakage, and eventually promote cell lysis. In some cases the AMP may be nonspecifically cytotoxic, which poses a hurdle for their clinical implementation. For example, human-derived LL-37 negatively affects keratinocyte viability at 10 melittin displays significant toxicity against epithelial cells at only 1.2 and FDA-approved nisin demonstrates toxicity to epithelial cells at 89.9 μM. To further understand how to reduce AMP toxicity for their use in the clinic, AMP mechanisms of action must be better understood.

Nosocomial infection plays a major role in causing failure of some biomedical devices. Each year in the United States, 5-10% out of 2 million fracture fixation devices and 1-2% of the 600,000 implanted joint prostheses become infected, leading to extended hospital stays and revision surgeries, ultimately costing over $250 million. The best way to combat these infections is to prevent bacterial colonization in the first place. Due to their broad spectrum activity and ability to be chemically manipulated, AMPs may provide a solution for a wide variety of applications including preventing the infection of medical devices. Tethering AMPs to medical devices may offer a promising way to prevent infection. However, there is still a need for a method of tethering AMPs to medical device that facilitates the anti-microbial function of the tethered AMP.

### SUMMARY

The present disclosure relates generally to anti-microbial peptides and medical device coatings including such peptides. In some embodiments, a coating composition comprises a tether covalently attached to an anti-microbial peptide, the tether having sufficient length to permit the anti-microbial peptide to at least partially penetrate a membrane of a bacteria, upon contact of the anti-microbial peptide with the bacteria. The anti-microbial peptide is derived from Chrysophsin-1, Chrysophsin-2, or Chrysophsin-3 by altering a terminal amino acid residue to covalently bind to the tether, for example peptides with the amino acid sequence of SEQ ID NO: 2, 3, 5, 6, 8 or 9. The tether can be a hetero-bifunctionalized polymer, with a first end being terminated with a N-hydroxysuccinimide (NETS) and a second end being terminated with a maleimide group to covalently bind the second end of the tether to the anti-microbial peptide. In some embodiments, the tether can have a length between about 15 nm and about 75 nm.

In some embodiments, a medical device comprises a surface; and an anti-microbial coating deposited on the surface, wherein the anti-microbial coating comprises: a tether covalently bound to the surface at a first end and covalently bound to an anti-microbial peptide at a second end, the tether having sufficient length to permit the anti-microbial peptide to at least partially penetrate a membrane of a bacteria, upon contact of the anti-microbial peptide with the bacteria. The surface of the medical device may be functionalized to facilitate binding of the tether to the surface.

In some embodiments, a method for preparing an anti-microbial medical device comprises applying a tether to a surface of a medical device to covalently attach the tether to the surface at a first end of the tether; and binding an anti-microbial peptide to a second end of the tether, where the tether is selected to have characteristics allowing the tether to maintain or promote an anti-microbial activity of the anti-microbial peptide. The surface of the medical device can be functionalized to enhance its binding properties.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 illustrates an AMP attached to a tether that has been bound to a surface;

FIG. 2 illustrates a typical quartz crystal microbalance with dissipation monitoring (QCM-D) response to grafting an anti-microbial peptide to a functionalized surface (in this case, silicon dioxide) via a tether where frequency (Δf) and dissipation (ΔD) are depicted on the primary and secondary y-axes, respectively, versus time, at the 3^{rd}, 5^{th}, 7^{th}, 9^{th} and 11^{th} overtones, and a-i represent time stamps according to changes in material flow through the QCM-D; a=tether flow, b=tether incubation, 30 min; c=buffer rinse, d=peptide flow, e=peptide incubation, 30 min, f=buffer rinse, g=bacteria flow, h=bacteria incubation, 1 hr, i=buffer rinse;

FIG. 3 shows the mass attachment (ng) of the tether molecules versus tether size according to QCM-D software models at different overtones;

FIG. 4 shows the thickness of the tether plus anti-microbial peptide layer (nm) according to QCM-D software models, compared to physically-adsorbed peptides;

FIGS. 5A-5B show the areal mass (ng/cm²) versus tether length calculated using the Voigt-Kelvin Model for tethered peptides via different tether sizes (FIG. 5A), and these areal mass values (solid bars) compared to the estimated values for each overtone using the Sauerbrey estimation (cross-hatched bars representing each overtone), (FIG. 5B);

FIG. 6 demonstrates bacterial mortality in relation to tether length against Gram-negative E. coli and Gram-positive S. aureus, calculated using the counts of live and dead fluorescent cells after a 1 hour exposure to peptides covalently bound to the surface for each tether length;

FIG. 7 demonstrates the proposed mechanism of activity of physically-adsorbed (A) and covalently bound (B-D) and anti-microbial peptides attached to various length tethers;

FIGS. 8A-8B present diagrams of water molecules in association with different PEG layers for PEG 2000 (FIG. 8A) and PEG 7500 (FIG. 8B);

FIG. 9 demonstrates one embodiment of a tethering process using the QCM-D method, over a surface (any surface) and depicts a flow order of the different components flown through the QCM-D instrument and over QCM-D sensors starting, from the top of the diagram to the bottom;

FIG. 10 shows data of turbidity (indicating degree of bacterial growth), measured via OD(590) demonstrating activity of a C-CHY1 (SEQ ID NO:1) coating on 1-cm segments, sectioned in cylindrical cross-sections, of Foley urinary catheters using PEG 866 and PEG 7500 tethers compared to bare surfaces and surfaces with only the APTMS functionalization, versus Pseudomonas aeruginosa (ATCC 29260), with increased OD(590) indicating increased bacterial growth; and

FIG. 11 shows additional OD(590) turbidity data, demonstrating activity of a C-CHY1 coating on Foley urinary catheter segment that has been prepared using an oxygen plasma cleaner to clean the surface and deposit hydrophilic oxygen molecules on the surface prior to functionalization and peptide attachment, with PEG 866 and PEG 7500 tethers compared to bare surfaces and surfaces with only the APTMS functionalization versus P. aeruginosa (ATCC 29260), with increased OD(590) indicating increased bacterial growth;

FIG. 12 shows the minimum inhibitory concentrations (MIC) of CHY1 (SEQ ID NO: 1), C-CHY1 (SEQ ID NO:2) and M-CHY1 (SEQ ID NO: 3) against several strains of bacteria, defined as the minimum concentration (μM) required to inhibit 100% visible growth of the microbe;

FIG. 13A and FIG. 13B demonstrate the cytotoxicity profile of M-CHY1 (SEQ ID NO: 3) anti-microbial peptide at different concentrations against a human primary fibroblast cell line, at 8 hours (FIG. 13A) and 30 hours (FIG. 13B) of exposure by measuring the fluorescence at 590 nm indicating the reduction of AlamarBlue® by healthy cells seeded at 4,000 cells per 96-well;

FIG. 14 shows the cytotoxicity profile of C-CHY1 (SEQ ID NO: 2) against a human primary fibroblast cell line after 12 hours of exposure to various concentrations of C-CHY1, measured using the OD(590) signal of the reduction of MTT into formazan after solubilizing in dimethylsulfoxide, and the dotted line indicates the average level of MTT reduction into formazan of cells without peptides;

FIGS. 15A-15B demonstrate the morphological characteristics of human primary fibroblasts when exposed to 5 μM (top), 10 μM (middle) and 30 μM (bottom) concentrations of M-CHY1 (SEQ ID NO: 3) (left panels) compared to untreated samples (right panels) at 10 hours' exposure (FIG. 15A) and 30 hours' exposure (FIG. 15B), taken using the 10× magnification of a Leica light microscope;

FIG. 16 demonstrates the optimization process of PEG 7500 linker molecules on the surface, by demonstrating the mass of PEG bound to the surface as a function of temperature (x-axis) and salt concentration (“high salt” is twice the salt concentration of normal PBS buffer), since PEG optimization is hypothesized to be directly related to resulting anti-microbial activity.

FIG. 17 demonstrates an example calculation of the percent dead S. aureus bacteria calculated after 1 hour exposure to C-CHY1 anti-microbial peptide tethered via PEG 7500 after temperature optimization of the PEG attachment, at 55 degrees C.

FIG. 18 illustrates a typical QCM-D response to grafting a M-CHY1 (SEQ ID NO: 3) anti-microbial peptide to a functionalized surface via a tether of PEG 7500, where frequency and dissipation are depicted on different axes versus time, at the 3rd, 5th, 7th, 9th and 11th overtones, and a-c represent major time stamps according to changes in material flow through the QCM-D; a=tether flow, b=peptide flow, c=bacteria flow.

FIG. 19 demonstrates a comparison of the killing ability of PEG 7500-tethered C-CHY1 and M-CHY1 anti-microbial peptides tethered to a surface against Gram-negative E. coli and Gram-positive S. aureus bacteria.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

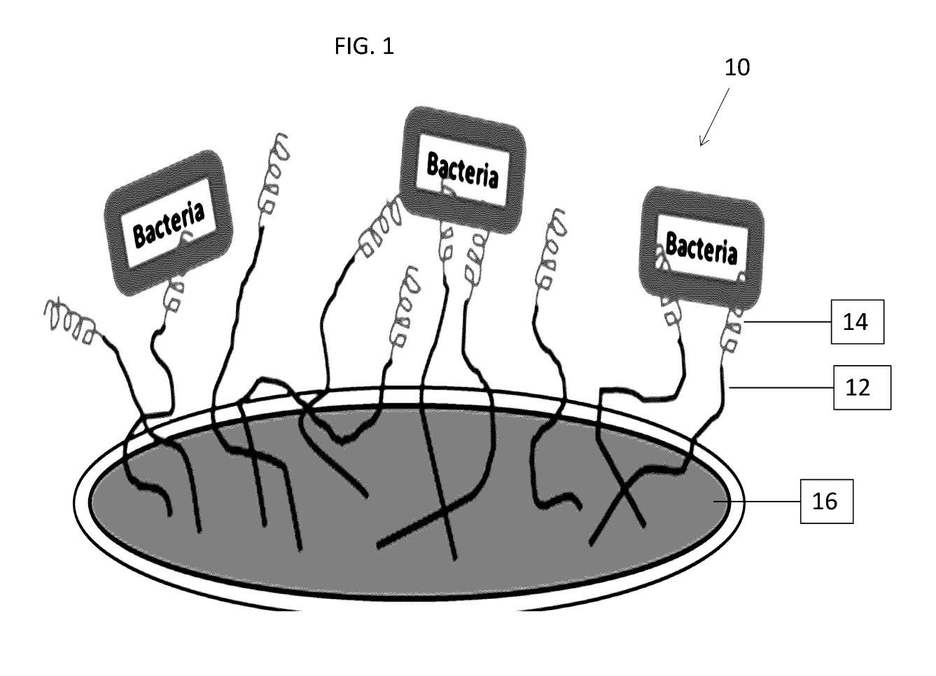

In reference to FIG. 1, in some embodiments, there is provided an anti-microbial composition 10 comprising a tethering molecule or tether 12 attached (such as by a covalent bond) to an anti-microbial peptide (AMP) 14. The composition 10 may be applied as a coating to a surface 16 of a device, such as, for example, a medical device, in need of anti-microbial protection.

In some embodiments, there is provided a composition comprising a tether covalently attached to an anti-microbial peptide, wherein the tether comprises a polymer that has sufficient flexibility and length allowing the polymer to maintain or promote an anti-microbial activity of the anti-microbial peptide when the tether is covalently attached to a surface. In some embodiments, the anti-microbial peptide may have a modified terminal end to facilitate binding of the anti-microbial peptide to the tether without interfering with the anti-microbial activity. In some aspects, there is disclosed an anti-microbial device comprising a surface with a silicone coating, a tether covalently attached to the surface, and an anti-microbial peptide covalently grafted to the tether. In some embodiments, the surface may be functionalized. In some embodiments, the anti-microbial peptide has a functional domain with an anti-microbial activity, and a tether binding domain, the tether binding domain being modified to allow for grafting to the tether. The tether comprises a polymer that has sufficient flexibility and a length allowing the polymer to maintain or promote an anti-microbial activity of the anti-microbial peptide when the tether is covalently attached to a surface. In some embodiments, the tether comprises polyethylene glycol (PEG). In some embodiments, the tether comprises maleimide polyethylene glycol N-hydroxysuccinimide ester molecules. In some embodiments, the anti-microbial peptide is selected from a group of peptides of SEQ ID NO: 1-SEQ ID NO: 9.

In some embodiments, there is disclosed an anti-microbial device comprising of a surface with a silicone coating, anti-microbial peptides, which may be chemically linked to the tether molecule and physically adsorbed onto the surface by means of natural diffusion and non-covalent interactions. In some embodiments, the surface retains the peptide and demonstrates effective anti-microbial activity. In some aspects, the anti-microbial peptide prevents the formation of biofilms. In some embodiments, biofilms are prevented by bactericidal action of the anti-microbial peptide on the surface. In some embodiments biofilms are prevented by the choice of tether molecule that prevents initial adhesion of bacteria to the surface.

AMPS.

Anti-microbial peptide (AMP) may be any peptide having an anti-microbial activity. In some embodiments, AMPs may be short peptide sequences (8-50 amino acids) and most are amphipathic and cationic, having a positive charge between about 2 and about 10. They are associated with the innate immunity of several species. Due to a continuous exposure to a diverse array of pathogens, AMPs of the present disclosure may have broad spectrum activity against bacteria, fungi, viruses, and protozoa. In some embodiments, AMPs may be selected for their potent activity against common Gram-negative and Gram-positive microbes, such as Escherichia coli (E. coli), Staphylococcus epidermidis, Pseudomonas aeruginosa (P. aeruginosa), Proteus mirabilis and Staphylococcus aureus (S. aureus), and activity against resistant organisms such as Methicillin-resistant S. aureus (MRSA), which are common in hospitals and community environments. The AMPs can act against the bacterial membrane with physical interaction, typically forming pores and causing cell lysis.

In some embodiments, AMPs will have a functional domain with an anti-microbial activity, and a tether binding domain opposite the functional domain where a terminal amino acid residue may be modified to allow for covalent bonding to the tether. In some embodiments, the modification may be the addition of a cysteine or methionine residue to either the N- or C-terminal end of the peptide. AMPs may be made without regard to stereochemistry, as the tethering process introduces natural resistance to proteolysis, but AMPs may be made in either the all D- or all L-isomeric forms if necessary to further inhibit (or even promote) proteolysis. D-isomeric amino acids are less susceptible to proteolytic degradation then their L-isomeric counterparts. Negatively charged amino acids such as aspartic acid (D) and glutamic acid (E) can be replace with positively charged amino acids such as arginine (R) histidine (H) or lysine (K). Higher positive charge is associated with more anti-microbial activity. Hydrophobic residues may be strategically substituted in order to change the helicity of the peptide which may affect the ability of the peptide to insert itself into bacterial membranes. The AMP can be between at least 70%, and in some embodiments, at least 85% pure, as determined by HPLC, otherwise the impurities may affect binding and anti-microbial activity. In some aspects, the anti-microbial peptide prevents the formation of biofilms by preventing initial bacterial adhesion and by its bactericidal action.

In some embodiments, AMPs will comprise an amino acid sequence derived from Chrysophsin-1 (CHY1; FFGWLIKGAIHAGKAIHGLIHRRRH; SEQ ID NO: 1). In some embodiments, CHY1 can be modified at the C-terminus or the N-terminus to facilitate covalent binding of the AMP to the tether, without effecting anti-microbial activity of CHY1. In some embodiments, AMPs of the present disclosure may include peptides having a modified terminal end while retaining the anti-microbial activity of CHY-1, and in some embodiments, the derivative may comprise an amino acid sequence with at least 95%, 90%, 85%, 80%, or 75% identity to SEQ ID NO: 1. In some embodiments, the N-terminus cysteine-modified Chrysophsin-1 (C-CHY1; CFFGWLIKGAIHAGKAIHGLIHRRRH; SEQ ID NO: 2) and the N-terminus methionine-modified Chrysophsin-1 (M-CHY1; MFFGWLIKGAIHAGKAIHGLIHRRRH; SEQ ID NO: 3) can be used. In some aspects, the modified peptides, including M-CHY1 and C-CHY1 may be toxic in high concentrations to human fibroblasts, above 10 μM concentrations. In some embodiments, these toxic concentrations may influence the appearance and morphology of the cells that have been exposed as a function of exposure time. In some embodiments, the modification, adding the cysteine (SEQ ID NO: 2) or methionine (SEQ ID NO: 3), is at the N-terminal end as the C-terminal end may include charged arginine residues. The charged arginine resides may allow for the anti-microbial activity of the Chrysophsin-1 peptide. Cysteine has a sulfhydryl group which allows for binding of the peptide to the spacer molecule. Methionine also has a sulphur group that allows for covalent binding to the tether. Chysophsin-1 and the modified Chysophsin-1 peptides, C-CHY1 and M-CHY1, are salt tolerant peptides helping retain their activity in the in vivo environment. Chysophsin-1 and the modified Chysophsin-1 peptides, C-CHY1 and M-CHY1, have significant positive charges at their C-terminal end due to the arginine (R) and histidine (H) localized at this end. Additional modifications having similar characteristics are possible. In some embodiments, isoforms of Chrysophsin-1 and their derivatives, Chrysophsin-2 (CHY2, SEQ ID NO: 4; FFGWLIRGAIHAGKAIHGLIHRRRH, C-CHY2, SEQ ID NO: 5; CFFGWLIRGAIHAGKAIHGLIHRRRH, M-CHY2, SEQ ID NO: 6; MFFGWLIRGAIHAGKAIHGLIHRRRH) or Chrysophsin-3 (CHY3, SEQ ID NO: 7; FIGLLISAGKAIHDLIRRRH, C-CHY3, SEQ ID NO: 8; CFIGLLISAGKAIHDLIRRRH, M-CHY3, SEQ ID NO: 9; MFIGLLISAGKAIHDLIRRRH) may be used. In some embodiments, AMPs of the present disclosure may include peptides having an amino acid sequence with at least 95%, 90%, 85%, 80%, or 75% identity to peptides having one of amino acid sequences SEQ ID NO: 1-SEQ ID NO: 9.

In some embodiments, the AMPs may comprise one or more AMPs selected from SMAP-29 (Sheep myeloid anti-microbial peptide), LL-37 (human derived cathelicidin peptide), EPI-1 (Epinecidin; derived from Orange Spotted Grouper), TP 3 (Tilapia piscidin 3), TP 4 (Tilapia piscidin 4), BmKn2 (derived from Scorpion), TET213 (synthetic), Cecropin P1 (derived from pigs), Cecropin A (derived from pigs), and Dermaseptin-59 (derived from Phyllomedusa sauvagei). In some embodiments, the AMPs may comprise derivatives of SMAP-29, LL-37, EPI-1, TP 3, TP 4, BmKn2, TET213, Cecropin P1, Cecropin A, and Dermaseptin-59.

Tethers.

The tether can be any substance capable of covalently binding an AMP to a surface of a device without interfering with the anti-microbial activity of the AMP. In some aspects, tethering the anti-microbial peptides covalently to surfaces may decrease their ability to interact with mammalian membranes but retain their anti-microbial activity. In some embodiments, the act of tethering may also increase peptide stability. The tether may be a polymer, and in some embodiments the polymer will have a flexibility and a length that will promote or maintain the anti-microbial activity of the AMP. In some embodiments, the tether is selected to have characteristics (flexibility, length, charge etc.) or other dominant factors that promote or maintain the peptide mechanism of action against bacteria. Flexibility of the tether may allow for the peptides to aggregate into clusters in order to form pores in the bacterial membrane. The flexibility of the tether may allow for the peptide to change its conformation to promote bacterial killing. The peptide may adopt a different mechanism of action when tethered. In some embodiments, the length of the tether is sufficient to allow for pore formation of the membrane. Flexible tethers typically do not have large pendant groups such as benzene, and the backbone is primarily composed of but not exclusively comprised of carbon-carbon single bonds. The tether may have sufficient length to allow for AMP interaction with the bacterial membrane as well as the ability of AMP to penetrate the membrane. Tether length could be used to control the specificity of anti-microbial action. Higher charges of the tether molecule may increase the affinity of the peptide for bacterial membranes, thus increasing the likelihood of the AMP to impart bactericidal action.

The tether may be a hydrophilic polymer such as PEG, a zwitterionic polymer, or a hydrophobic polymer, as long as such tethers are sufficiently flexible. For zwitterionic polymers the overall charge of the polymer is neutral although the backbone components may carry a net positive or negative charge. In some embodiments, tethers may carry a net positive or net negative charge and which can be dependent upon the number of monomer units. For the monomer the charge can be between −3 and +3 at neutral pH.

In some embodiments, flexibility of a polymer refers to the ability of the polymer chain to bend and flex to allow for lateral movement of the peptide relative to the surface and aggregate especially in the presence of bacteria. Flexible polymers generally have a low glass transition temperature as well as a backbone structure that typically does not include large pendant groups or a benzene ring in the main chain without sufficient carbons in-between, which may increase stiffness. This flexibility may be desirable especially for longer chains. In some embodiments, the flexibility of the tether may allow the AMP to aggregate on the bacterial cell membrane and adopt their native pore-forming mechanism.

In some embodiments, length of a polymer may refer to the maximum possible extension of the tether and can be directly related to the thickness of the anti-microbial coating. For example for PEG 866, its maximum length is 5.2 nm and PEG 7500 is 58 nm. Higher molecular weight (longer) PEG chains are more desirable as they allow for better interaction and penetration of the bacterial membrane. The length of the tether may be sufficient to allow for anti-microbial action of the tethered peptide. The molecular weight of the tether will vary depending upon the polymer. For example, in some embodiments, a tether having a length between about 5 nm and about 100 nm can be used. In some embodiments, a tether having a length between about 25 nm and 75 nm may be used. Depending of the length of the AMP 1 a tether having a length between about 15 nm and 75 nm can be sufficient.

If a tether has a linear, non-branched chain, length is directly related to the molecular weight of the chain. In some embodiments, polymers with molecular weight between about 800 g/mol and about 15000 g/mol can be used as a tether. For branched or hyper-branched polymers this would not be the case. Examples of such polymers include, but is not limited to, PEG with 3 or more functional arms, one for binding to the surface and two or more for the peptide, which can allow for increased AMP binding, leading to higher AMP densities which can improve anti-microbial activity, PEG based dendrimers with functionality depending on the number of generations, in some embodiments, between 1 and 4 generations. For such polymers, higher molecular weights are desirable, between 5000 g/mol and 100,000 g/mol. The use of branched or hyper branched polymers as tethering molecules may allow for additional functional groups and potentially higher peptide surface densities. This allows for more functional groups at the end of the branches to which the modified AMPs can bind. Increased AMP density can improve anti-microbial efficacy.

In some embodiments, length of the polymer may be sufficient for the attached AMP to interact with the bacterial membrane and form pores. The molecular weight of the tether will vary depending upon the polymer. For example, in some embodiments, a tether having a length between about 5 nm and about 100 nm can be used. In some embodiments, a tether having a length between about 25 nm and 75 nm may be used. Depending of the length of the AMP a tether having a length between about 15 nm and 75 nm can be sufficient.

In some embodiments, the tether may have a shorter length where charge density is more important than length for the peptide mechanism of action and is the dominant factor for anti-microbial activity. The importance of charge density depends upon the AMP. For example for C-CHY1 the charge density may be more important for anti-microbial activity than tether length at PEG with a molecular weight of less than 2000 g/mol and less than 20 nm in length. For other peptides and tethers, this will vary.

In some embodiments the tether will be biocompatible and the length and composition may be selected to minimize fouling. Hydrophilic polymers such as PEG have been shown to reduce protein and bacterial adsorption onto surfaces. This may be due to their strong interaction with water molecules making it energetically unfavourable for proteins to bind to the surface to the exclusion volume, entropic effects and steric repulsion. There needs to be a sufficient density of the PEG, or hydrophilic polymer, this density varies by length of the polymer. Zwitterionic and hydrophobic polymers work by minimizing surface energy. By combining the protein and bacterial resistant properties of the tether with the anti-microbial activity of the AMP, an antifouling surface may be prepared.

In some embodiments, the tether can be hetero-bifunctionalized, with one end of this being terminated with a N-hydroxysuccinimide (NETS) and the other end being terminated with a maleimide group. The maleimide end group allows for attachment of the N- or C-terminal modified anti-microbial peptide by formation of a thioether bond. Such tether can also bind to a medical device surface via displacement of the NHS group by the amide group. In some embodiments, the tether may comprise maleimide PEG N-hydroxysuccinimide ester molecules (MAL-PEG-NHS). The NHS binds to a surface and the maleimide end group allows for attachment of the N- or C-terminal modified anti-microbial peptide by formation of a thioether bond. MAL-PEG-NHS may have molecular weights (MW) between about 800 Da and about 15,000 Da, for example, 866, 2000, or 7500 (referred to as PEG 866, PEG 2000 and PEG 7500, respectively, and collectively PEG). In some embodiments the PEG will have a MW of from about 750 to about 10,000. In some embodiments, the PEG will have a MW from about 800 to about 8,000.

In some embodiments, the polymer can be a polycation or polycation functionalized with the maleimide and NHS chemistry described herein. In some embodiments, the polymers may be polyethylene, polyurethane/s, polylactide-co-glycolic acid, dendritic polymers or zwitterionic compounds. The molecular weight and length of these polymers, polycations or polyanions may be adjusted to provide desired properties, such as, for example, flexibility and length. In some embodiments a tether may not be needed and the modified peptides C-CHY1 and M-CHY1 can be physically adsorbed to the device surface.

In some aspects, tethers may be reacted with the anti-microbial peptide prior to addition onto a functionalized surface, or prior to its physical adsorption onto the surface by means of natural diffusion and non-covalent interactions. In some embodiments, the surface retains the peptide and demonstrates effective anti-microbial activity. In some embodiments, the peptide comprises of one or more AMPs having the amino acid sequence of SEQ ID NO: 1, 2, 3, 4, 5, 6, 7, 8, 9, and 10.

In some embodiments biofilms are prevented by the choice of tether molecule that prevents initial adhesion of bacteria to the surface.

Coatings.

In some embodiments, the above compositions of AMPs bound to a tether may be prepared as a coating. In some embodiments, the tethers and AMPS may be provided in a solvent, such as, for example, phosphate buffered saline (PBS) or PBS supplemented with between 1 and 5 mM ethylenediaminetetraacetic acid (PBS/EDTA). Other solvents can be used, as well as additional additives.

In some embodiments the coating may be supplied separately and may be applied as a coating to a surface of a device at the point of use. In some embodiments, the coating may be applied to the device at the time of manufacturing of the device. In some embodiments, the surface may be functionalized. In some embodiments, device is a medical device. In some embodiments the medical device may be, for example, a catheter such as a urinary, central venous, peripherally-inserted or central catheter. In some embodiments the medical device may be an orthopaedic device such as an artificial hip or knee, or may be spinal implants, orthopaedic pin or screw. In some embodiments, the surface may be on a surgical instrument such as scalpels, forceps, tweezers, and sutures. In some embodiments, the coating may be applied to any surface where inhibition of bacterial growth in a medical or health care setting is desires.

In some embodiments, the coating may be applied to the surface of a commercial device, consumer product or article of manufacture in order to inhibit biofilm formation or bacterial growth. The commercial device or article of manufacture may be used for food storage and preparation or packaging, such as, for example, plastic wrap or meat packaging. In some embodiments the surface is a surface found on cutting boards and counters, or on processing equipment such as batch mixers, homogenizers, and grinders. In some embodiments the consumer product may be baby and children's toys such as pacifiers and rattles, or paints that may be used on boats, pools, and decks.

In another aspect of the present disclosure, a device is provided wherein the coating has already been applied to the device.

The device may be a medical device. In some embodiments, the medical device will be, for example, a catheter such as a urinary, central venous, peripherally-inserted or central catheter. In some embodiments the medical device is an orthopaedic device such as an artificial hip or knee, or may be spinal implants, orthopaedic pin or screw. In some embodiments, the surface may be on a surgical instrument such as scalpels, forceps, tweezers, and sutures.

In certain embodiments, biofilms are prevented by bactericidal action of the anti-microbial peptide coated on the surface.

In some aspects, there is disclosed a method for preparing an anti-microbial medical device, the method comprising covalently attaching a tether to the functionalized surface; and binding the tether to an anti-microbial peptide, where the tether is selected to have characteristics allowing the tether to maintain or promote an anti-microbial activity of the anti-microbial peptide. In some embodiments, the method may further comprise a step of functionalizing the surface of the medical device to facilitate covalent binding of the tether.

In some embodiments the surface may be functionalized to enhance the covalent binding of the coating to the surface of a device. The tether provides a covalent link between the AMP and the functionalized surface with the tether separating the two. The functionalized surface may be a surface of a medical device, such as, for example, a Foley catheter. The surface to be functionalized is cleaned, using ethanol, deionized water, sodium dodecyl sulphate (SDS) follow by deionized water and then oxygen plasma cleaned. The surface to be functionalized may be functionalized using 3-aminopropyltrimethoxysilane (APTMS), by coating the surface with APTMS, onto which a tether is attached. In some embodiments, the tether can be hetero-bifunctionalized, with one end of this being terminated with a N-hydroxysuccinimide (NHS) and the other end being terminated with a maleimide group. Such tether can bind to the APTMS via displacement of the NHS group by the amide group. The maleimide end group allows for attachment of the N- or C-terminal modified anti-microbial peptide by formation of a thioether bond. This process works with any anti-microbial peptide with a cysteine or methionine end group. Other processes for functionalizing the surface may be utilized, such as spray deposition, electrospinning, free radical initiation (grafting from), with the initiator being a peroxide (as an example benzyol peroxide (BPO)) using a grafting from technique then capping with an end group that has a maleimide group.

The functionalizing step may comprise submerging the medical device in a 10% (v/v) 3-(aminopropyl)trimethoxysilane in methanol solution and rinsing the medical device with methanol and deionized water to yield a functionalized medical device. Other functional groups may be used such as 3-(aminopropyl)triethoxysilane, or those that allow deposition of free amine groups on the surface. The grafting step further comprises incubating the medical device with the tether for a period of time sufficient to attach the tether covalently to the functionalized surface to yield a coated medical device. The grafting of the tether can be performed between 20° C. and 70° C., with 23° C., 37° C., 45° C., 50° C. and 55° C. optimally used for binding. The optimal binding temperature may vary depending on the tether molecular weight (MW), PEG MW=866, MW=2000 and MW=7500, named PEG 866, PEG 2000 and PEG 7500, respectively, and surface functionalization. The number of monomer PEG molecules that comprise each is defined by this molecular weight, and the length of each PEG molecule is defined by the number of PEG monomers. The tether can be adhered to the surface for a period of 30 minutes up to 1 hour. The tether comprises a polymer that has a flexibility and a length allowing the polymer to maintain or promote an anti-microbial activity and anti-biofilm activity, of an anti-microbial peptide when the tether is covalently attached to the surface.

In some embodiments, the device will have a surface that may be silicone-coated and functionalized to allow for the grafting of a tether to the surface, where the tether has a flexibility and a length to promote or maintain an anti-microbial function of an AMP, and an AMP will be attached to the tether where the AMP has an anti-microbial domain and a tether binding domain. In some embodiments, the present method include the steps of providing a medical device with a silicone-coated surface, cleaning the medical device with ethanol, functionalizing the silicone-coated surface, covalently grafting a tether to the functionalized medical device, rinsing the coated medical device with a buffer, and incubating the coated medical device with the anti-microbial peptide wherein the incubating allows the tether to covalently bind to the anti-microbial peptide.

Determining Thickness of Coating.

The overall thickness of the coating may be determined using quartz-crystal microbalance with dissipation monitoring (QCM-D). QCM-D may allow calculation of mass deposition, provided that the deposited material is sufficiently ridged, according to the specifications in the Sauerbrey or Voigt-Kelvin models. QCM-D may allow determination of the thickness of the coating to be determined in real time. The overall thickness may be determined using QCM-D using a combination of parameters such as mass deposition or peptide surface density and theoretical calculations. When thickness is measured using the QCM-D, this allows calculation of grafted layer thickness (first the tether layer thickness followed by the tether plus peptide layer thickness). This allows calculation of the tether length when it is tethered to the surface in real-time. In some embodiments, the proper thickness of the coating may be determined using alternative methods, such as ellipsometry and atomic force microscopy.

The range of desired thicknesses for the tether molecule may be more than about 5 nm, for example, between about 15 nm to about 75 nm, or about 20 nm to about 75 nm. In some embodiments, it may be between about 40 nm and about 65 nm. The overall thickness of the system combined with the AMP can depend on the number of amino acids and folding properties (i.e. globular versus linear versus helical) of the peptide when tethered on the surface.

To accommodate several peptide mechanisms, specificities, and desired orientations of molecules within the coating, various combinations of tethers and/or peptides may be combined within the same coating. This will allow for tailoring of the surface to polymicrobial infiltration (i.e. mixtures of Gram-positive and Gram-negative bacteria or species of fungi), which may allow for the adoption of several peptide mechanisms with one coating. The thickness of such a heterogeneous surface can vary with location along the device, and thus the average thickness may be calculated using several methods, such as QCM-D, ellipsometry and atomic force microscopy.

Further the QCM-D can allow calculation of a surface density of the material grafted onto the surface.

Bacteria can be introduced into the QCM-D in real-time to study the anti-microbial action of covalently tethered anti-microbial peptides. Killing percent (also called bacterial mortality) can be determined using microscopy, ex situ from the QCM-D, to determine mechanisms, and related back to surface density.

The QCM-D is a flow process and thus can have a step-by-step procedure to study the covalent tethering of various molecules.

QCM-D measures changes in frequency (Δf) and dissipation (ΔD) that correspond to changes in mass deposition (Δm) and film rigidity, respectively, on the surface of oscillating piezoelectric quartz crystal sensors with nanogram-level sensitivity. It is a non-destructive flow technique, allowing for coupling to other experiments. QCM-D can be used to show appropriate tethering of AMP to the surface of a device.

Based on QCM-D data, layer thickness (nm) and grafting density (ng/cm²) can be calculated using the Voigt-Kelvin viscoelastic model. Thickness of layers is positively correlated with increasing tether length. For example, PEG 866 and 7500 demonstrate the shortest and longest layer thicknesses, respectively. The layer with PEG 7500 is only 38% extended compared with layers formed with PEG 866 and PEG 2000, which are both greater than 75% extended. This is consistent with the formation of a “mushroom like monolayer” of PEG 7500, where the chains are tangled. In some embodiments, the mushroom-like monolayer provides a slightly porous network for swelling caused by water and surrounding media, allowing for the incorporation of other small molecules that can be released from the monolayer, such as, for example, ionic groups, antibiotics and other small molecule drugs, or growth factors.

In some embodiments, the monolayer may prevent protein adhesion. In some embodiments, the density of the molecules on the surface of the device will lead to tangling, providing less physical space for debris or bacteria to reach the device surface and begin colonizing it. In some embodiments, the tether and AMP density will be high enough to reduce this type of fouling. Further, the mushroom-like monolayer provides a slightly porous network for swelling caused by water and milieu. This polymer swelling could allow for the incorporation and release of other small molecules.

Larger (i.e. longer or increased molecular weight) tethers may interpenetrate due to the favourable thermodynamics of entanglement and self-interaction. In some embodiments, the larger tethers may take up more space because neighbouring chains intertwine. In some embodiments, the larger tether length (for example, PEG 7500) allows the AMP to have higher activity than the medium tether length (for example, PEG 2000).

Grafting.

In some embodiments, tether and AMP layers are assembled using a “grafting to” technique. “Grafting to” techniques allow strict control of tether length by the addition of whole tether versus “grafting from” techniques, where the addition of single monomers at a time leaves a relatively heterogeneous brush in terms of length. Dead bacteria, or the bacterial membrane debris, may remain adhered to the tethered anti-microbial peptide surface due to charge interactions. The bacteria may also remain adhered if there is no flow (in applications not in the blood stream or urine which may help rinse the surface). In some embodiments, the tether may act like a brush and help remove bacterial debris mechanically from the surface extending the useful life of the coating. In some embodiments, grafting the longest tether may lead to steric hindrance that limits not only tether grafting to the surface but also the number of binding sites available for the AMP, which may enhance the activity of the tethered AMP. Other techniques for assembling the layer may be utilized, such as such as free radical initiation, with the initiator being a peroxide (as an example benzyol peroxide (BPO)) using a grafting from technique then capping with an end group that has a maleimide group.

Tether Optimization.

Changing temperature of the tether incubation may reduce steric hindrance of the longer tether spacers (for example, PEG 2000 and PEG 7500) to allow for higher density binding on the surface. This can allow for more peptide binding including (C-CHY1) binding to the surface. For example, temperatures between about 20° C. and about 70° C. may be used to increase binding of the heterobifuntionalized PEG molecules, which can increase the anti-microbial activity of the result tethered anti-microbial peptide.

In some embodiments, M-CHY1 may be adsorbed and bound to the surface using QCM-D to demonstrate similar broad-spectrum activity as C-CHY1 bound via the same length of linker molecule.

In some embodiments, AMPs can be covalently tethered with the tether to indwelling medical devices, such as Foley urinary catheters, to provide a potential solution to preventing medical device-associated infection due to their broad spectrum activity, low likelihood of bacterial resistance, and anti-microbial activity. The tether length, AMP surface density, and AMP flexibility on tethered may influence the anti-microbial activity of the present compositions. In some embodiments described herein tethers of various lengths are used to graft AMPs to functionalized devices. QCM-D is used to calculate thickness (nm) and density (ng/cm²) of a tethered AMP, which may be CHY-1 or C-CHY1, with different PEG tether lengths, and to relate these properties to anti-microbial activity.

The systems and methods of the present disclosure are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Examples

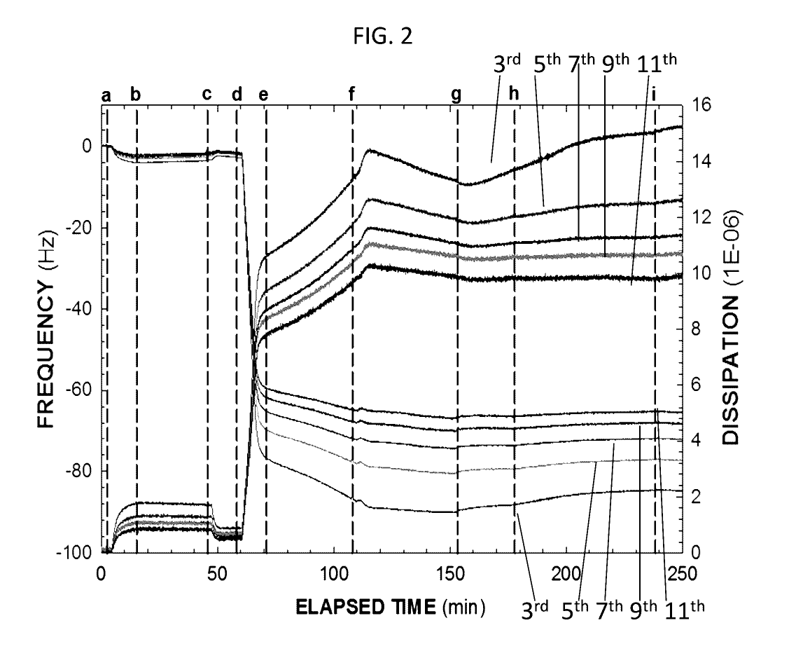

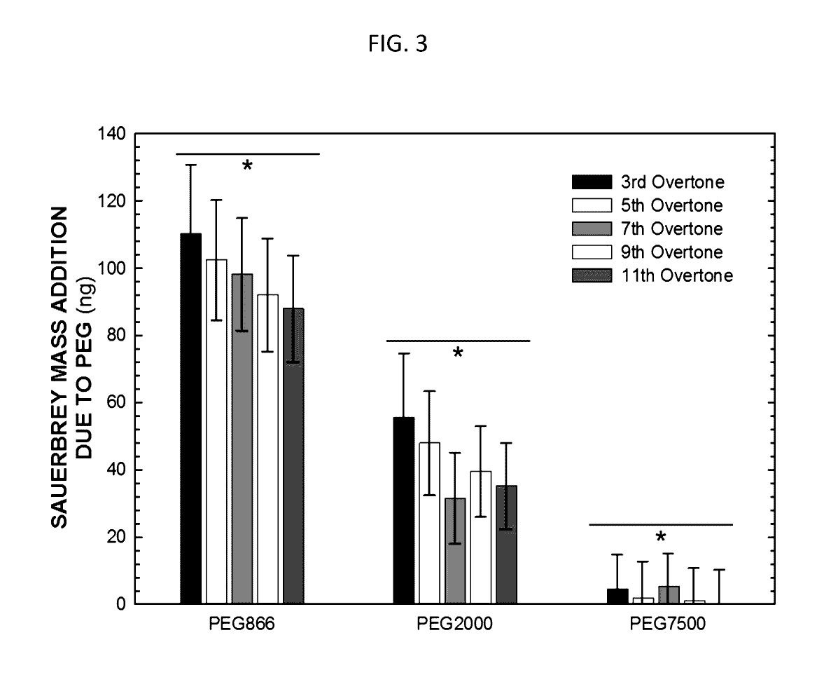

Referring to FIG. 1, the surface is functionalized with the anti-microbial peptide via a flexible linker molecule and exposed to bacteria in the QCM-D using QCM-D sensors as the substrate. Referring to FIG. 2 QCM-D was used to monitor covalent attachment in real-time of the PEG spacer molecule followed by introduction of the C-CHY1 peptide to the functionalized SiO₂ surface (FIG. 2). According to the Sauerbrey calculation, the Δm due to PEG spacers is between 2 and 100 ng per crystal (Table 1, below; FIG. 3). The mass of PEG attached decreases with increasing tether length (FIG. 3). PEGylated surfaces show a rigid surface (ΔD values from +1×10⁻⁶ to +2×10⁻⁶ Hz) compared to the bulk fluid baseline, validating the use of Eqn. 1 and 2 for PEG layer calculations. When C-CHY1 is introduced, there is a rapid decrease of Δf corresponding to a Δm of 0.8 to 2.2 μg (Table 1) bound to the surface. This step is accompanied by a large increase in ΔD from near +2×10⁻⁶ to +20×10⁻⁶ Hz after the introduction of C-CHY1. Comparatively, CHY1 introduction leads to a Δf decrease corresponding a Δm of about 0.4 μg of CHY1 being physically-adsorbed on the surface. During the rinse, a slightly higher increase in Δf of +5 Hz suggests some of the CHY1 is removed, consistent with its non-covalent nature, but about 50% of the peptide remains adsorbed. The ΔD does not deviate from the baseline value, suggesting that a rigid surface is maintained throughout the entire CHY1 experiment.

TABLE 1
Sauerbrey and Voigt-Kelvin Calculations of Mass Attachment,
Layer Thickness and Areal Mass for each PEG Tether Length

| | | | | PEG/C- | | |
|---|---|---|---|---|---|---|
| ^{a}Type | PEG | | | CHY1 | C-CHY1 areal mass | |
| of | attachment | Chrysophsin-1 attachment | | layer | (ng/cm²) | |
| tether | ng | ng | | thickness | | ^{c}Voigt- |
| molecule | ^{b}Sauerbrey | ^{b}Sauerbrey | ^{c}Voigt-Kelvin | (nm)^{c} | ^{b}Sauerbrey | Kelvin |
| No | — | 204.9 ± 7.99 | 373.3 ± 58.6 | 2.50 ± 0.49 | 260.9 ± 10.2 | 475.3 ± 74.6 |
| Tether | | | | | | |
| (PHYS- | | | | | | |
| ADSORB) | | | | | | |
| PEG | 98.1 ± 7.88 | 874.0 ± 48.8 | 835.2 ± 168 | 6.59 ± 1.14 | 1113 ± 62.2 | 1063 ± 215 |
| 866 | | | | | | |
| PEG | 41.8 ± 6.77 | 2238 ± 60.4 | 2284 ± 113 | 17.6 ± 1.32 | 2850 ± 76.9 | 2909 ± 144 |
| 2000 | | | | | | |
| PEG | 2.46 ± 4.58 | 1753 ± 43.9 | 1870 ± 176 | 21.9 ± 1.86 | 2232 ± 55.0 | 2381 ± 224 |
| 7500 | | | | | | |

^{a}All values represent the mean for the 3^{rd} through 11^{th} overtones. And error reported represents standard error for n > 3 surfaces total, corrected for sample size by dividing standard deviation by the square root of the number of replicates.

Once anti-microbial peptide is grafted, the layer becomes significantly more dissipative as demonstrated by raw QCM-D data (FIG. 2), which has been seen in previous work. Thus, parameters of the system were modeled using the Voigt-Kelvin extended viscoelastic model in Q-Tools software (Biolin Scientific, Stockholm, Sweden). This model corrects the Sauerbrey estimations for higher energy dissipation by adding terms to the Δf relation to mass (Eqn. 3) and ΔD relation to film rigidity (Eqn. 4).

$$\begin{matrix}Δ f = - \frac{{η}_{L}}{2 {πδ}_{L} {m}_{q}} - {f}_{o} \frac{{m}_{f}}{{m}_{q}} [ 1 - \frac{2}{{ρ}_{f}} {( \frac{{η}_{L}}{{δ}_{L}} )}^{2} \frac{{G}^{″}}{{G}^{′2} + {G}^{″2}} ] & ( Eqn . 3 ) \\ Δ D = \frac{{η}_{L}}{n π {f}_{o} {δ}_{L} {m}_{q}} + \frac{{m}_{f}}{{m}_{q}} [ \frac{4}{{ρ}_{f}} {( \frac{{η}_{L}}{{δ}_{L}} )}^{2} \frac{{G}^{′}}{{G}^{′2} + {G}^{″2}} ] & ( Eqn . 4 )\end{matrix}$$

Where η_{L} is the viscosity of the bulk liquid assumed to be water (kg/m·s); δ_{L}, and δ_{f} are the decay lengths of the acoustic wave in the bulk liquid and film (m), respectively; m_{q} and m_{f} are the (kg), respectively; ρ_{f} is the film density (kg/m³). The layer was modeled to get numerical outputs for layer viscosity, density, and shear modulus (ηₘ, ρₘ, and μₘ), and film thickness. All overtones were modeled at once. The bulk liquid, predominantly PBS or PBS/EDTA, was assumed to have the same viscosity and density as water at 23° C. Model step size and output ranges were changed based on calculated theoretical values using estimated extended molecule size, and the lowest chi square value (χ²) was taken. Once values of thickness (nm) and density (kg/m³) were found with the model, the two were multiplied to calculate areal mass in ng/cm².

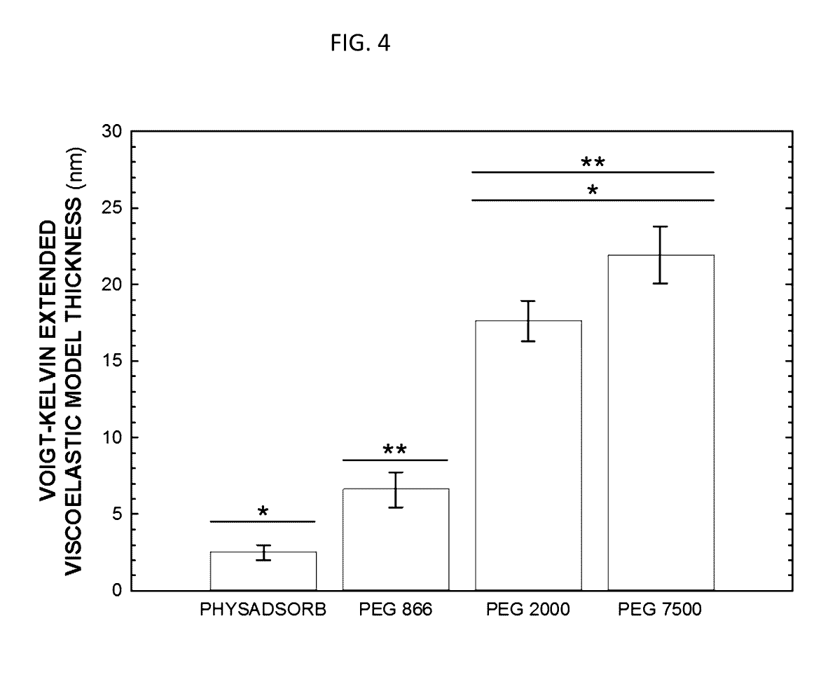

FIG. 4 demonstrates a measure of the thickness (nm) of the covalently-bound tether and anti-microbial peptide (C-CHY1) film on the crystal surface modelled using the Voigt-Kelvin model (Table 1; FIG. 4; Eqns 3 and 4). A theoretical maximum thickness using the assumption of the full extension of PEG and C-CHY1 was calculated using molecular bond lengths and found to be 8.8 nm 28, 17.8 nm and 58.05 nm for PEG 866, 2000 and 7500, respectively. Tethered C-CHY1 via PEG 866 is approximately 6.59±1.14 nm thick. As expected this thickness increases with increasing PEG size, from 17.6±1.32 nm for PEG 2000 and 21.9±1.86 nm for PEG 7500. These numbers suggest that the PEG was almost fully extended for PEG 866 and 2000 because of their similarity to the calculated values. For PEG 7500, the modelled thickness is 38% of theoretical maximum thickness, suggesting that the PEG is not fully extended. This could be due to PEG 7500 chains interacting with neighbouring chains and peptides, entangling with each other. Thickness of the physically-adsorbed CHY1 film was similarly modelled. The thickness is 2.5±0.5 nm.

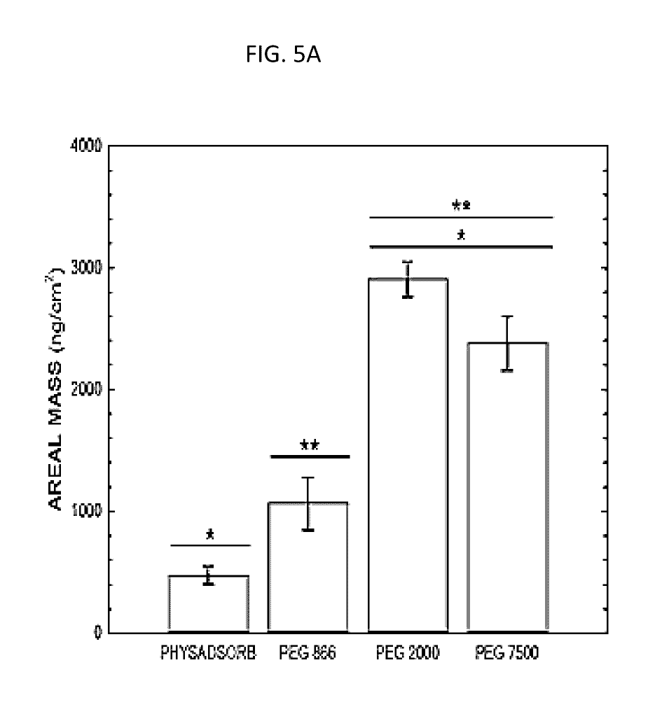

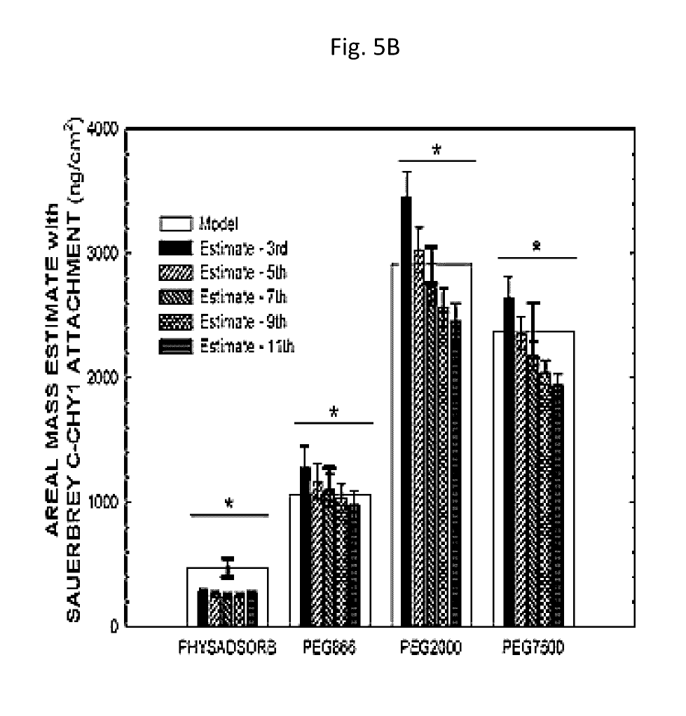

Referring to FIG. 5A and FIG. 5B for each of the PEG spacer lengths, the areal mass was determined using the density (kg/m³) obtained by the Voigt-Kelvin viscoelastic model. The areal masses calculated for n=3 replicates were 1063±215 ng/cm², 2909±144 ng/cm², and 2381±223 ng/cm² for PEG 866, 2000 and 7500, respectively (Table 1; FIG. 5A). This areal mass includes the mass of the PEG spacer, attached C-CHY1, and the trapped buffer solution. Under the Voigt-Kelvin model, the following assumptions are made: a Newtonian bulk fluid, a laterally homogenous and evenly distributed film, a soft and viscoelastic film (high ΔD), and an adsorbed layer is coupled to the sensor. This model utilizes the ΔD and Δf values contributed by the entire film on the surface, including associated buffer molecules, to determine thickness and density (Eqns 3 and 4 above).

For comparison, the Sauerbrey equation was used to calculate the mass addition between PEG flow and bacteria flow to find the overall grafted mass at each overtone. Then, this was divided by the crystal surface area (FIG. 5B). This estimation was a good fit for the areal mass of grafted PEG, peptide and associated water. To ensure this good fit, the model and estimated areal masses were compared (FIG. 5B). The dense packing of PEG 866 demonstrated in FIG. 3 limits its trapped buffer molecules to those associated with PEG monomers only. Thus, using the total system mass and the mass of PEG monomer-associated water molecules was also a good fit for lower tether lengths (FIG. 5B).

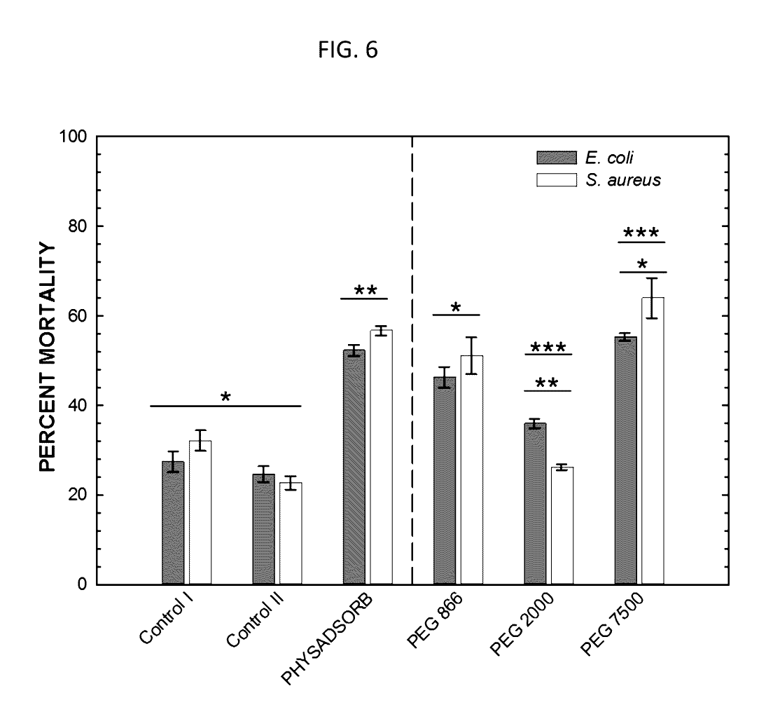

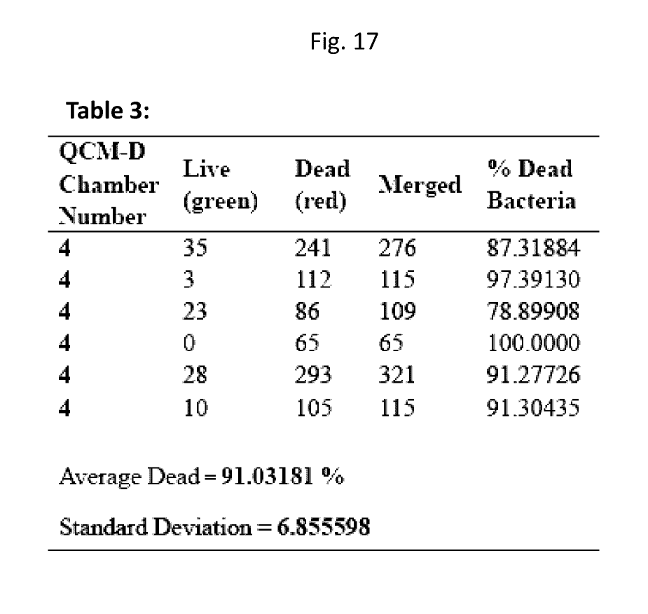

FIG. 6 demonstrates that longer tether length shows highest antimicrobial activity. For E. coli, the highest activity was achieved with the longest spacer length used, PEG 7500 (FIG. 6). For the shortest spacer PEG 866, 46±2.3% mortality of E. coli was achieved. Similarly, the highest activity of C-CHY1 against S. aureus was achieved with the longest spacer length, PEG 7500, at 64±4.5% mortality (FIG. 6) at 23° C. Using PEG 7500 binding at 55° C., changing the temperature back to 23° C. for C-CHY1 binding and S. aureus incubation, 91±6.9% mortality was achieved (FIG. 17).

The physically-adsorbed CHY1 (FIG. 6) caused 52.3±1.2% and 56.7±1.1% mortalities of E. coli and S. aureus, respectively.

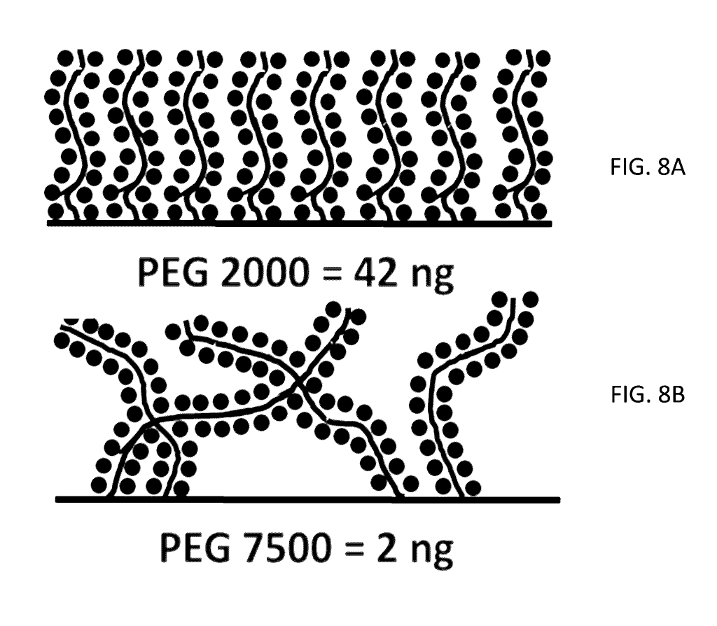

Sauerbrey calculations suggest that fewer PEG molecules attach as tether length increases, implying that there is also increased spacing between them. With increased spacing, more water molecules may be trapped within PEG chains and thus, more contributed mass. Indeed, Voigt-Kelvin modeling of raw QCM-D data demonstrated higher areal masses for both PEG 2000 and PEG 7500 than PEG 866 (Table 1). Areal mass due to PEG 2000 was higher than that of PEG 7500. Even though there are considerably more water molecules associated with one PEG 7500 molecule compared with one PEG 2000 molecule (FIG. 8), there are significantly more PEG 2000 molecules (FIG. 8A, 42 ng total) compared to PEG 7500 (FIG. 8B; 2 ng total) leading to a higher areal mass.

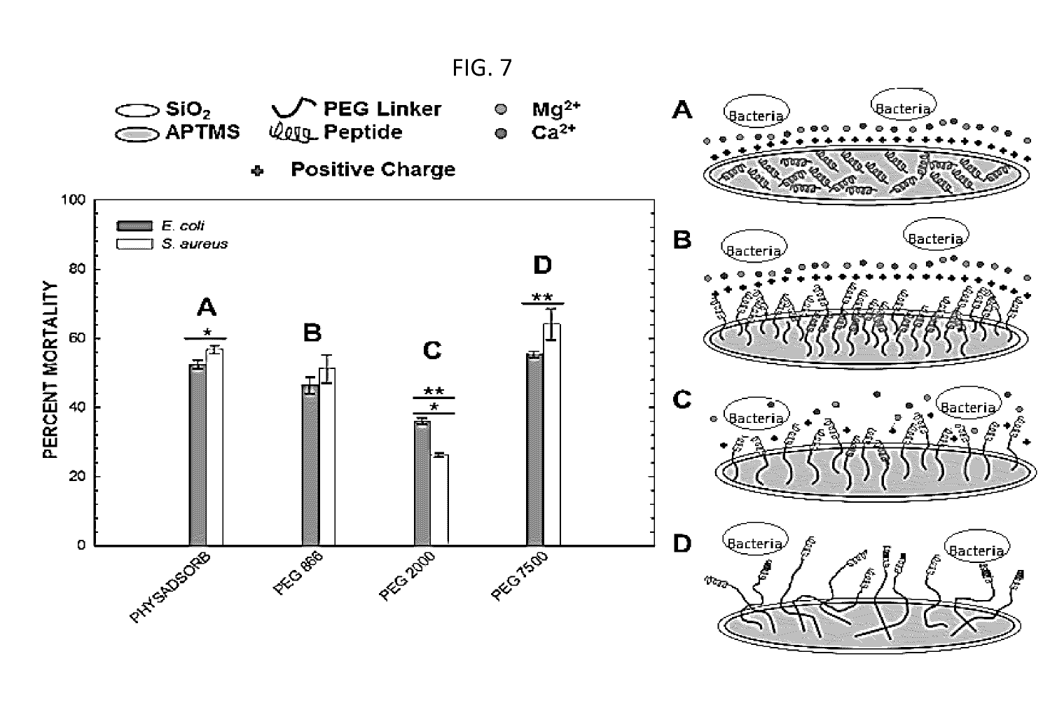

In solution, CHY1 has been shown to act against bacterial membranes through pore formation, as demonstrated by QCM-D and other techniques. Tethered C-CHY1 activity was not seen to be dependent on bacteria type, but was influenced by tether length and peptide surface density, as similar trends with respect to PEG properties were seen for E. coli and S. aureus. This agrees with the identical MICs determined of C-CHY1 against both strains. The ability of QCM-D to characterize layer thickness and density in a non-destructive manner allowed coupling of these results with the results of anti-microbial assays to determine C-CHY1 mechanisms of anti-microbial action for physically adsorbed peptide (FIG. 7A) and each tether length (FIG. 7B-D).

In some embodiments described herein, CHY1 is physically adsorbed to a device, in which there was no tether. A high local charge density could cause an alternative mechanism of anti-microbial activity to pore formation, as seen in other studies, involving the displacement of positive cations from the membranes of both E. coli and S. aureus (FIG. 7A). The calculated areal mass suggests that there is 5 times more CHY1 on the surface than what has previously been observed (Table 1). It is possible that the lower density of CHY1 is close to the minimum charge density threshold for activity, where increasing charge density increases anti-microbial activity.

For the shortest tethered system with PEG 866, its thickness is only 6.49±1.14 nm which does not physically allow the peptide to span the E. coli or S. aureus membranes, which are 23 and 80 nm thick, respectively. Despite this, there is still anti-microbial activity, suggesting an alternative mechanism to pore formation (FIG. 7B). This mechanism is also consistent with the QCM-D findings. The high density of PEG 866 molecules (FIG. 3) leads to a high density of C-CHY1 on the surface and thus a high local charge density. Higher charge densities cause stronger ionic displacement in bacterial membranes; thus, PEG 866-tethered C-CHY1 causes membrane destabilization due to displacement of positive cations from bacterial membranes, ultimately leading to cell death (FIG. 7B).

FIG. 7 further demonstrates that C-CHY1 tethered to PEG 2000 has the lowest ability to kill either bacterial strain, suggesting that the PEG tethering interferes with anti-microbial activity. The thickness of this system, 17.6±1.3 nm, suggests that the spacer is 98% extended but is still not long enough to fully penetrate either membrane. Partial insertion of C-CHY1 into the membrane is possible, but this would limit the extent to which lysis and cell death occur. Despite the inability of PEG 2000-tethered C-CHY1 to adequately form pores, some activity still results, likely from a different mechanism. The comparatively low bacterial mortality of C-CHY1 with PEG 2000 versus PEG 866 tethers is due to lower charge density of C-CHY1 on the surface, since bactericidal activity has been shown to increase with increasing charge density (FIG. 7C). The lower charge density is due to steric hindrance between grafted PEG 2000 molecules leading to fewer binding sites available for C-CHY1 (FIG. 3).

In some embodiments, C-CHY1 is tethered to PEG 7500. This grafting exhibits the most efficient bactericidal activity against both strains of bacteria, showing that longer tethers demonstrate higher activity. Despite the lowest amount of PEG on the surface, lowest binding site availability, and the least peptide grafted onto the surface (FIG. 3 and FIG. 5), its long length provides enough thickness (FIG. 4) to fully penetrate the bacterial membranes and form pores, similar to how the peptide acts in solution. This thickness, however, is only 38% of its maximum theoretical extension, suggesting that the PEG chains in the system are entangled when there are no bacteria present. It is possible that C-CHY1 extension changes in the presence of bacteria due to changes in local charge allowing it to aggregate and then form pores. Many studies suggest that pore formation does not begin until a sufficient peptide-to-lipid ratio of aggregated peptides on the surface of the membrane is achieved. Thus, the proposed mechanism of action is pore formation followed by lysis and cell death, thus allowing for the highest activity across all tether lengths (FIG. 7D).

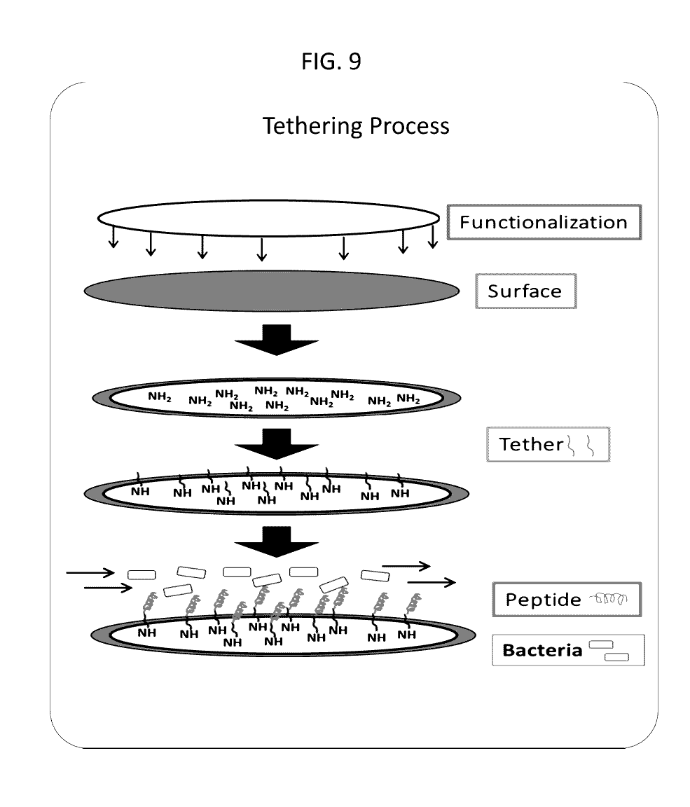

FIG. 9 demonstrates a tethering process using the QCM-D instrument and its sensors as the substrate. However, methods of tethering other than QCM-D, for example, dip-coating, may be utilized in this order. First a surface is cleaned, then the surface is silanized by submerging the surface in a 10% (v/v) (3-aminopropyl)-trimethoxysilane (APTMS) in methanol solution for 20 min. Following that the surface is then rinsed with methanol and DI water yielding a functionalized surface. A bi-functionalized polyethylene glycol (PEG) with maleimide and N-hydroxysuccinimide ester functional end groups is then introduced (MAL-PEG-NHS) to the functionalized surface. The NHS binds to the functionalized surface and the maleimide end group allows for attachment to an N or C-terminally modified anti-microbial peptide, such as Chrysophsin-1, by formation of a thioether bond. MAL-PEG-NHS may have molecular weights (MW), 866, 2000, or 7500 (referred to as PEG 866, PEG 2000 and PEG 7500, respectively, and collectively PEG).

Materials and Methods

Bacterial Strains and Culturing. Escherichia coli HB101 (ATCC 33694) and Staphylococcus aureus (ATCC 48366) were cultured overnight in Luria-Bertani broth (20 g/L). For QCM-D and toxicity studies, cells were harvested at the late logarithmic phase in their growth curve, as confirmed by absorbance measurements (OD(600)=0.7-1.0 arbitrary units) (Thermo Scientific USA, Waltham, Mass. USA). The cells were centrifuged at 1284×g (Centrific Thermo Scientific, Waltham, Mass. USA) and re-suspended in 0.01 M, pH 7.2 phosphate buffered saline (PBS) (Sigma Aldrich, St. Louis, Mo. USA) twice, and then diluted 100-fold to approximately 10⁸ cfu/mL.

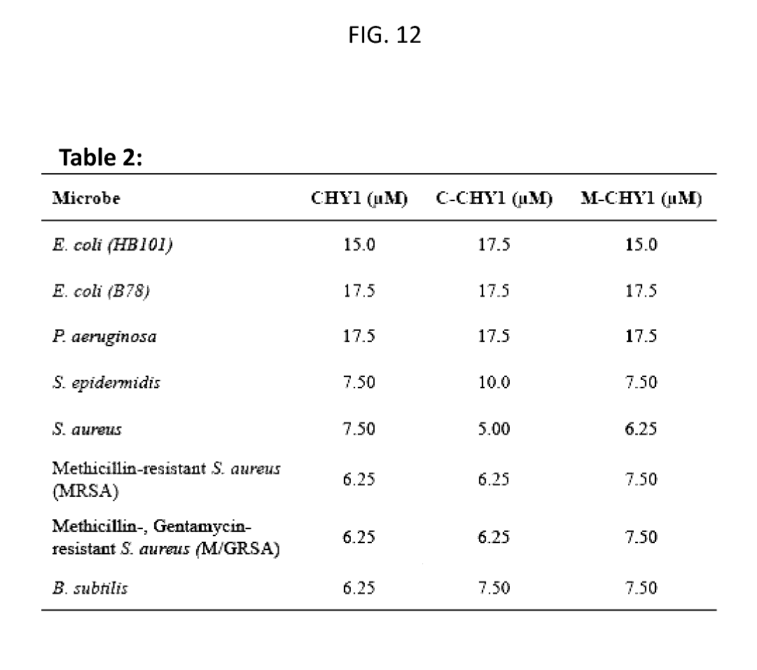

Peptides. Chrysophsin-1 (CHY1; FFGWLIKGAIHAGKAIHGLIHRRRH), the N-terminus cysteine-modified Chrysophsin-1 (C-CHY1; CFFGWLIKGAIHAGKAIHGLIHRRRH) and the N-terminus methionine-modified Chrysophsin-1 (M-CHY1; MFFGWLIKGAIHAGKAIHGLIHRRRH) were purchased from Bachem Americas, Inc. (Torrance, Calif. USA). The peptides were received as a lyophilized trifluoroacetate salt at greater than 85% purity confirmed by high performance liquid chromatography. Solutions of 5 g/L CHY1 and C-CHY1 were made in PBS (pH 7.2) and PBS supplemented with between 1 and 5 mM ethylenediaminetetraacetic acid (PBS/EDTA; pH 7.2) as a chelating agent respectively, and stored at −20° C. All buffer solutions for storage, dilutions and experimentation were degassed by sonication under vacuum for 30 min prior to their use. The minimum inhibitory concentrations (MIC) of these peptides against several common microbes were found prior to tethering (FIG. 12).

QCM-D: Covalent Linking of C-CHY1. SiO₂-coated quartz crystal sensors from Biolin Scientific (Stockholm, Sweden) were used as immobilization platforms for the modified C-CHY1. Before use, SiO₂ sensors were cleaned in the QCM-D at 40° C. using ethanol, DI water, 2% sodium dodecyl sulfate (w/v), DI water again, and then nitrogen dried. Lastly, sensor surfaces were treated for 2 min using an oxygen plasma cleaner (SPI Supplies, PA USA) to both clean and functionalize the surface. The SiO₂ crystals were then silanized by submerging in a 10% (v/v) (3-Aminopropyl)-trimethoxysilane (APTMS) in methanol solution for 20 min. Each sensor was then rinsed twice thoroughly with methanol and DI water and placed in each QCM-D chamber.

Changes in frequency (Δf, Hz) and dissipation (ΔD, ×10-6 Hz) at the 3rd, 5th, 7th, 9th and 11th overtones were monitored at a constant 23° C. in all chambers using a Q-Sense E4 QCM-D system (Biolin Scientific, Stockholm, Sweden). All flow rates for the solutions were at 0.1 mL/min unless otherwise noted, and all volumes given are on a per chamber basis. PBS/EDTA (pH 7.2) buffer was used to establish a stable baseline measurement. Maleimide PEG N-hydroxysuccinimide ester molecules (MAL-PEG-NHS) with molecular weights (MW) of 866 (ThermoScientific, Waltham Mass., USA), 2000, or 7500 (JenKem Technology USA Inc., Allen, Tex. USA) were purchased. These will be referred to as PEG 866, PEG 2000 and PEG 7500, respectively. One mL of 100 μM MAL-PEG-NHS was flowed through the QCM-D and subsequently incubated for 30 min. Crystals were then rinsed with 1.2 mL of the PBS/EDTA (pH 7.2) buffer. Similarly, 1.25 mL of 10 μM C-CHY1 solution was then flown over the sensors and allowed to incubate for 30 min. To rinse excess C-CHY1 off the surface and prepare for the introduction of 2 mL bacteria, a 45 min PBS rinse at 0.3 mL/min was first flowed through the QCM-D. The dilute bacterial solution was allowed to incubate for 1 hour, followed by a final 2 mL PBS (pH 7.2) rinse. The crystals were removed from the chambers and placed in individual petri dishes containing 0.85% (w/v) NaCl solution for bacterial viability testing.

QCM-D Control Experiments: Physical Adsorption of CHY1. Similarly, SiO₂-coated sensors were used for physical adsorption of unmodified CHY1 and were prepared as described above. For this experiment there was no APTMS functionalization or PEG flow, thus, after cleaning, the crystals were placed immediately into the QCM-D, PBS (pH 7.2) was used to establish a baseline and 1.25 mL of 10 μM CHY1 solution was introduced. Then, flow was stopped and CHY1 was incubated for 30 min. The rest of the protocol described in the previous section was then followed, including the 0.3 mL/min PBS rinse, bacterial flow, bacterial incubation and final PBS rinse. The crystals were removed from the chambers and placed in individual petri dishes containing 0.85% (w/v) NaCl solution for bacterial viability testing. For the next type of control, crystals that had never been coated with APTMS were cleaned and placed into the QCM-D chambers without functionalization. Starting with a PBS rinse, the crystals were exposed to bacteria solution for 1 hour and then rinsed before imaging. For the APTMS control, crystals were cleaned and functionalized with APTMS. Then the procedure continued starting with a PBS rinse (pH 7.2), bacteria introduction, incubation, and a final rinse before imaging. For the PEG control, the crystals were cleaned and functionalized with APTMS. A baseline was established, PEG was flown and then incubated. Peptide was not introduced in this type of control experiment. Separate PEG control experiments were performed for each PEG size. All experiments were repeated at least three times.

Bacterial Mortality. Bacterial mortality was determined immediately after the final rinse of the QCM-D experiment. Crystals were stained using a LIVE/DEAD BacLight Bacterial Viability Kit (Life Technologies Corp, NY USA) in 2 mL of 0.85% (w/v) NaCl solution with 5 μM SYTO 9 and 30 μM propidium iodide for 15 min. The crystals were rinsed once using 0.85% (w/v) NaCl solution to remove any excess dye and then kept in 1 mL saline to keep hydrated for imaging. The crystals were imaged at 20× objective using fluorescein isothiocyanate (526 nm) and Texas Red filters (620 nm) under a Nikon Eclipse E400 fluorescence microscope (Melville, N.Y. USA). A minimum of 5 locations on each crystal were examined for live and dead bacteria, totaling at least 10 images. Images were analyzed using ImageJ Software (http://rsbweb.nih.gov/ij/) to produce a merged image from which the percent mortality (or killing percent) of the cells was determined.

QCM-D: Data Modelling. The viscoelasticity of deposited material demonstrated by the QCM-D raw data was used to determine which model to use in estimating parameters of the system such as mass of attachment (ng), layer thicknesses (nm) and peptide areal mass (ng/cm2). The brush may be thought to be made up of two layers, first is APTMS plus PEG and second is C-CHY1. The former demonstrates near-zero dissipation values, thus, the Sauerbrey equation for rigid surfaces (Eqn. 1) applies, where Δm is inversely related to Δf. For the Sauerbrey model, ΔD and film rigidity are related using Eqn. 2.

$$\begin{matrix}Δ f = - \frac{2 {f}_{0}^{2}}{A \sqrt{{ρ}_{q} {μ}_{q}}} Δ m & ( Eqn . 1 ) \\ Δ D = \frac{{G}^{′}}{2 π {G}^{″}} & ( Eqn . 2 )\end{matrix}$$

Where f0 is the fundamental frequency of the quartz crystal, 5 MHz; A is the active crystal surface area; ρq is the density of quartz, 2.648 g/cm3; and μq is the shear modulus of the crystal, 2.947×1011 g/cm·s2; G″ is the loss modulus and G′ is the storage modulus of the film attached to the crystal surface. Thus, decreases in Δf demonstrate an addition of mass and higher ΔD values indicate a softer film. The Sauerbrey model was applied to time points from the flow of PEG through the flow of C-CHY1. A “maximum” PEG attachment was also calculated using the minimum frequency value between the two time points for comparison.

Once C-CHY1 is grafted, the layer becomes significantly more dissipative as demonstrated by raw QCM-D data, which has been seen in previous work. Thus, parameters of the system were modelled using the Voigt-Kelvin extended viscoelastic model in Q-Tools software (Biolin Scientific, Stockholm, Sweden). This model corrects the Sauerbrey estimations for higher energy dissipation by adding terms to the Δf relation to mass (Eqn. 3) and ΔD relation to film rigidity (Eqn. 4).

$$\begin{matrix}Δ f = - \frac{{η}_{L}}{2 {πδ}_{L} {m}_{q}} - {f}_{o} \frac{{m}_{f}}{{m}_{q}} [ 1 - \frac{2}{{ρ}_{f}} {( \frac{{η}_{L}}{{δ}_{L}} )}^{2} \frac{{G}^{″}}{{G}^{′2} + {G}^{″2}} ] & ( Eqn . 3 ) \\ Δ D = \frac{{η}_{L}}{n π {f}_{o} {δ}_{L} {m}_{q}} + \frac{{m}_{f}}{{m}_{q}} [ \frac{4}{{ρ}_{f}} {( \frac{{η}_{L}}{{δ}_{L}} )}^{2} \frac{{G}^{′}}{{G}^{′2} + {G}^{″2}} ] & ( Eqn . 4 )\end{matrix}$$

Where η_{L} is the viscosity of the bulk liquid assumed to be water (kg/m·s); δL and δf are the decay lengths of the acoustic wave in the bulk liquid and film (m), respectively; mq and mf are the (kg), respectively; ρf is the film density (kg/m3). The layer was modelled to get numerical outputs for layer viscosity, density, and shear modulus (ηm, ρm, and μm), and film thickness. All overtones were modelled at once. The bulk liquid, predominantly PBS or PBS/EDTA, was assumed to have the same viscosity and density as water at 23° C. Model step size and output ranges were changed based on calculated theoretical values using estimated extended molecule size, and the lowest chi square value (χ2) was taken. Once values of thickness (nm) and density (kg/m3) were found with the model, the two were multiplied to calculate areal mass in ng/cm2. The Sauerbrey estimate (Eqn. 1 and 2) for the QCM-D crystal surface area for the grafted C-CHY1 layer was used to compare with modelled values.

Because the long grafted layers are thought to be associated with water and the QCM-D modelling does not account for this, reported literature values of associated water molecules with PEG monomers were used to model the mass of the system accounting for water.

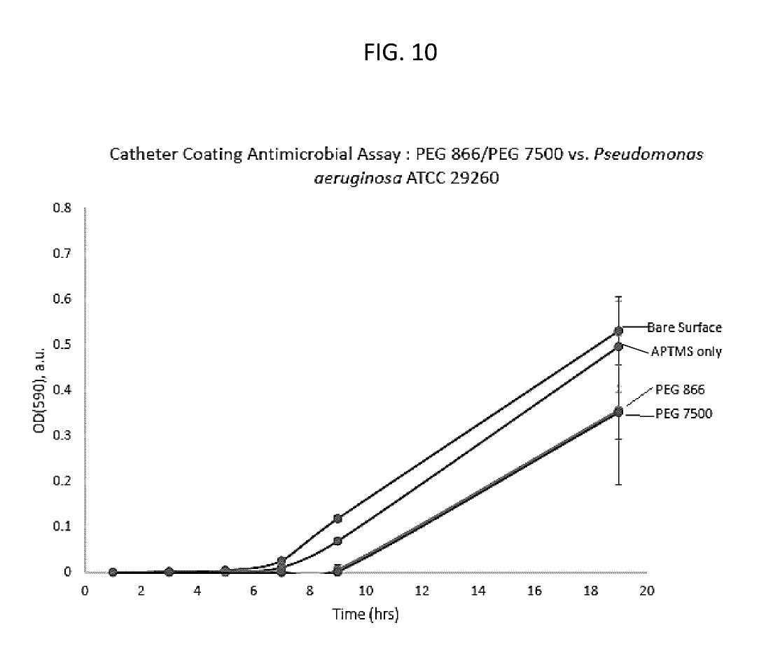

FIG. 10 shows data of turbidity (indicating degree of bacterial growth), measured via OD(590) demonstrating activity of a C-CHY1 coating on Foley urinary catheter segments using PEG 866 and PEG 7500 tethers versus Pseudomonas aeruginosa (ATCC 29260); and

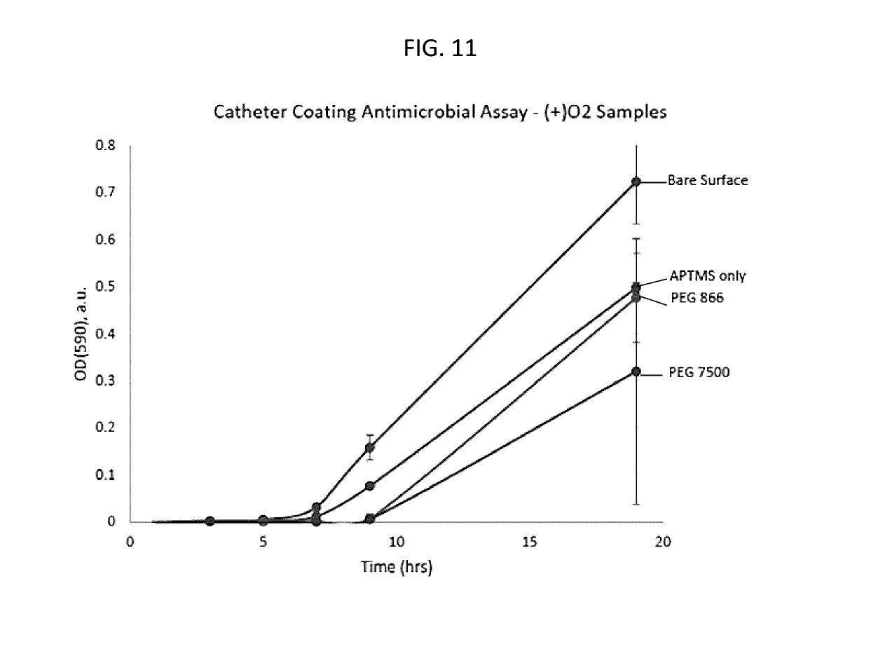

FIG. 11 shows additional data demonstrating activity of a C-CHY1 coating on Foley urinary catheter segment that has been prepared using an oxygen plasma cleaner to clean the surface and deposit hydrophilic oxygen molecules on the surface prior to functionalization and peptide attachment, with PEG 866 and PEG 7500 versus P. aeruginosa (ATCC 29260). To gather the data for FIG. 10 and FIG. 11, Foley catheter segments were sectioned in cylindrical segments, 1-cm long under sterile conditions. Segments were functionalized using a dip-coating method according to the procedure in FIG. 9, entered into 24-well plates and exposed to 2 mL MHB with 2×10⁵ CFU/mL bacteria at 37 degrees C. at 200 RPM agitation. At several time points between 30 minutes and 20 hours, 1 mL aliquots were taken from the each well and the OD(590) was measured. The 1 mL aliquots were returned to the respective wells and the plate was returned to 37 degrees C. and incubated until the next time point.

FIG. 12 shows Table 2 with the minimum inhibitory concentrations (MIC) of CHY1 (SEQ ID NO: 1), C-CHY1 (SEQ ID NO: 2) and M-CHY1 (SEQ ID NO: 3) against several strains of bacteria, at 16 hours after inoculation, defined as the minimum concentration (μM) required to inhibit 100% visible growth of the microbe. All values represent the mean from three experimental replicates. The MICs were determined using previously established protocols.

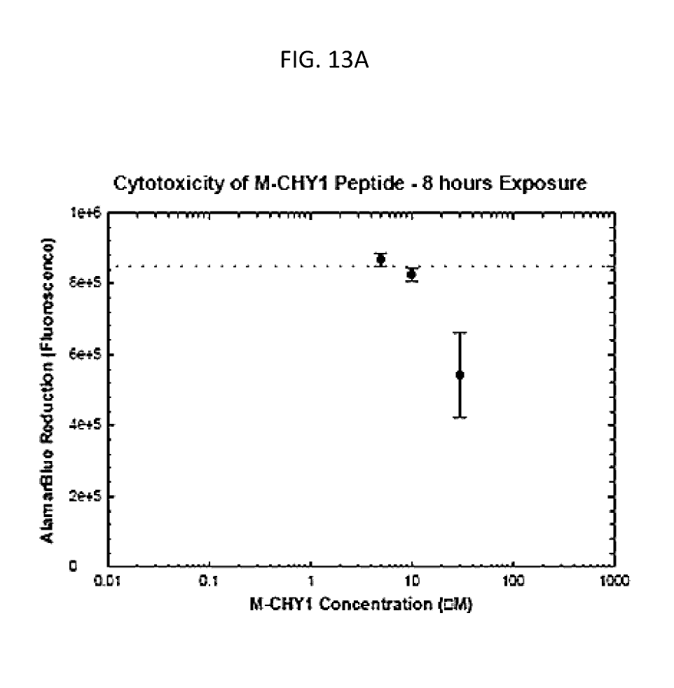

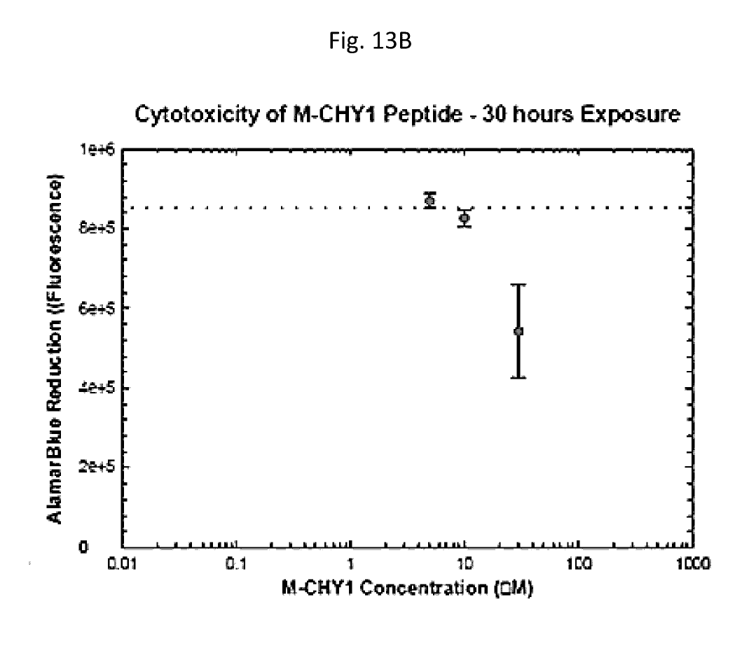

FIGS. 13A-13B demonstrate the cytotoxicity profile of M-CHY1 (SEQ ID NO: 3) anti-microbial peptide at different concentrations against a human primary fibroblast cell line, at 8 hours (FIG. 13A) and 30 hours (FIG. 13B) of exposure using the reduction of AlamarBlue® by healthy cells seeded at 4,000 cells per 96-well. The fluorescent signal at 590 nm was determined after seeding the cells overnight (12-16 hours), exposing the cells to peptide concentrations for 4 hours, and the addition of 10% v/v AlamarBlue® reagent for an additional 4 hours. (Total incubation with peptides, 8 hours), followed by medium and peptide refreshing and another addition of AlamarBlue® at 30 hours' incubation. All values represent mean±S.D. from at least three experimental replicates. The dotted line represents the average cell-only fluorescence at 590 nm. The AlamarBlue® procedure was followed according to manufacturer's instructions.

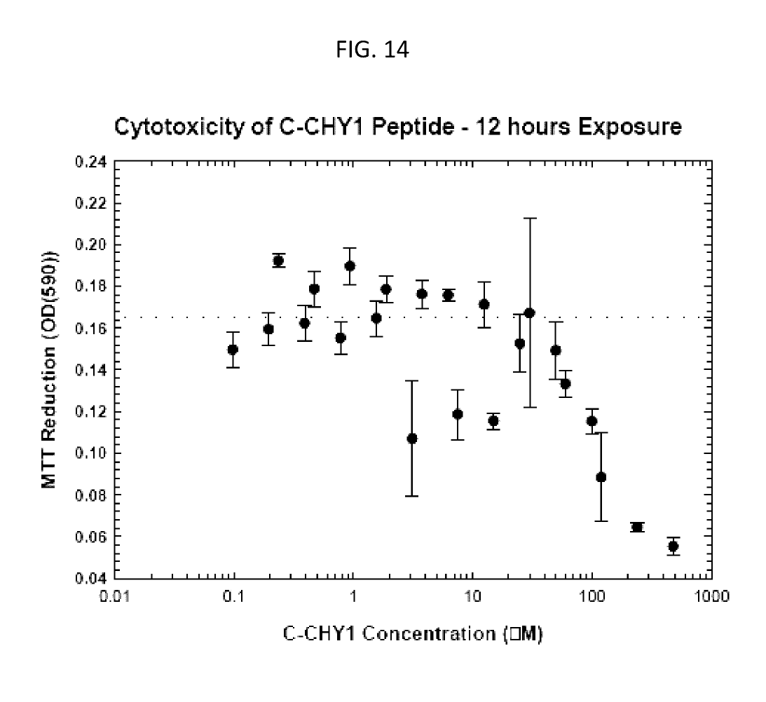

FIG. 14 show the cytotoxicity profile of C-CHY1 (SEQ ID NO: 2) against a human primary fibroblast cell line after 12 hours of exposure to various concentrations of C-CHY1, measured using the reduction of 3-(4,5-dimethylthiazol-2-yl)-2,5-diphenyltetrazolium bromide (MTT) into formazan solubilized by dimethylsulfoxide. Average optical density at 590 nm of MTT reduced by 4,000 cells per well of CT 1005 primary human fibroblasts, when exposed to various C-CHY1 peptides concentrations in solution after 12 hours of exposure. All values represent mean±S.D. from at least three experimental replicates. The dotted line represents the average cell-only MT reduction signal at 590 nm. The MTT procedure over 4,000 cells per well was followed according to manufacturer's instructions.

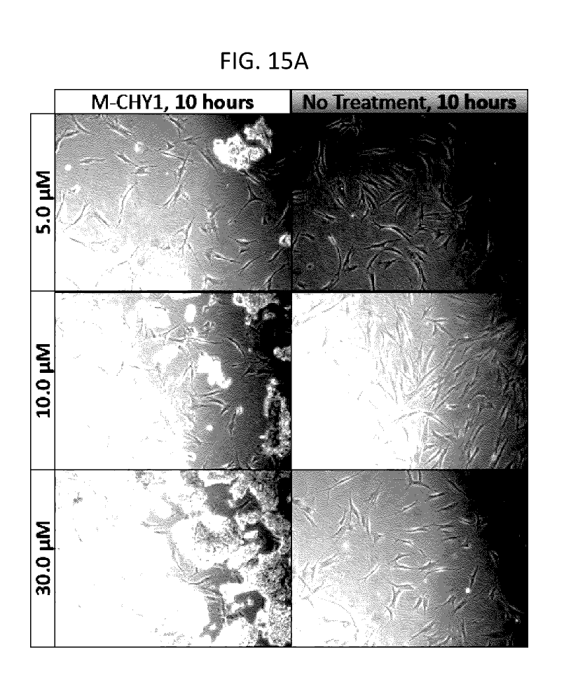

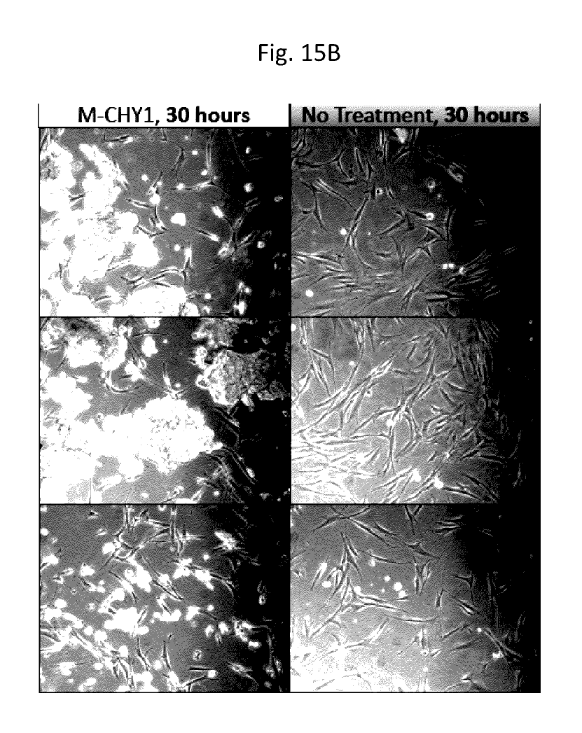

FIGS. 15A-15B demonstrate the morphological characteristics of human primary fibroblasts when exposed to 5 μM (top), 10 μM (middle) and 30 μM (bottom) concentrations of M-CHY1 (SEQ ID NO: 3) (left panels) compared to untreated samples (right panels) at 10 hours' exposure (FIG. 15A) and 30 hours exposure (FIG. 15B), taken using the 10× magnification of a Leica light microscope. Photographs were taken during AlamarBlue® treatment described and depicted in FIG. 13A and FIG. 13B.

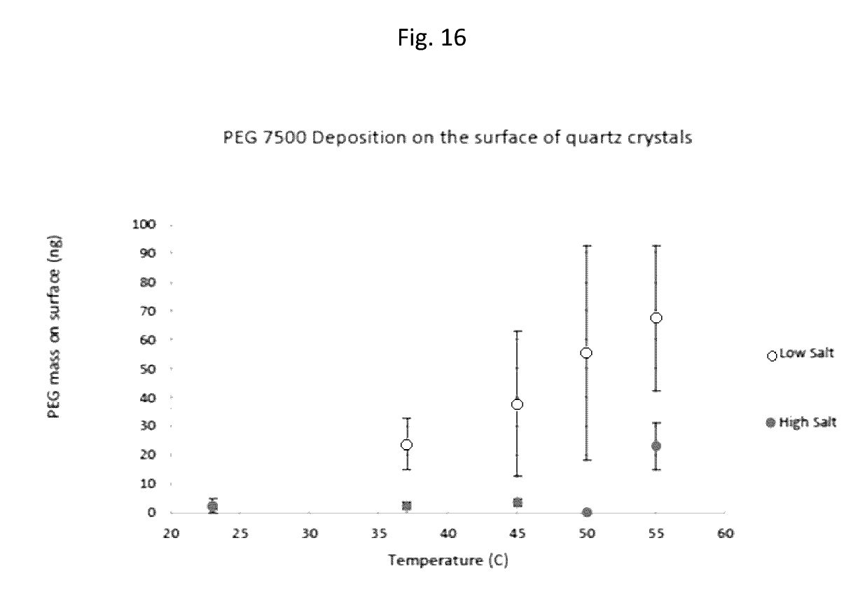

FIG. 16 demonstrates the optimization process of PEG 7500 linker molecules on the surface, by demonstrating the mass of PEG bound to the surface as a function of temperature (x-axis) and salt concentration (“high salt” is twice the salt concentration of normal PBS buffer), since PEG optimization is hypothesized to be directly related to resulting anti-microbial activity. As temperature increases, the amount of PEG 7500 increases. This may be due to the change of conformation of PEG in solution and on the surface due to the higher temperatures reducing steric hindrance allowing for more binding of the PEG. Higher than physiological salt concentrations (“high salt”) alters the binding ability of PEG 7500 linker to bind.

FIG. 17 included table 3, which is an example calculation of the percent dead S. aureus bacteria calculated after 1 hour exposure to C-CHY1 anti-microbial peptide tethered via PEG 7500 after temperature optimization of the PEG attachment, at 55 degrees C. Temperature was reduced to room temperature prior to C-CHY1 binding and bacteria introduction, and bacterial mortality was calculated by counting the number of live and number of dead bacteria in different areas on the QCM-D sensor (columns 2 and 3), merging the images and counting the live bacteria (column 4) and calculating the percent dead (column 5).

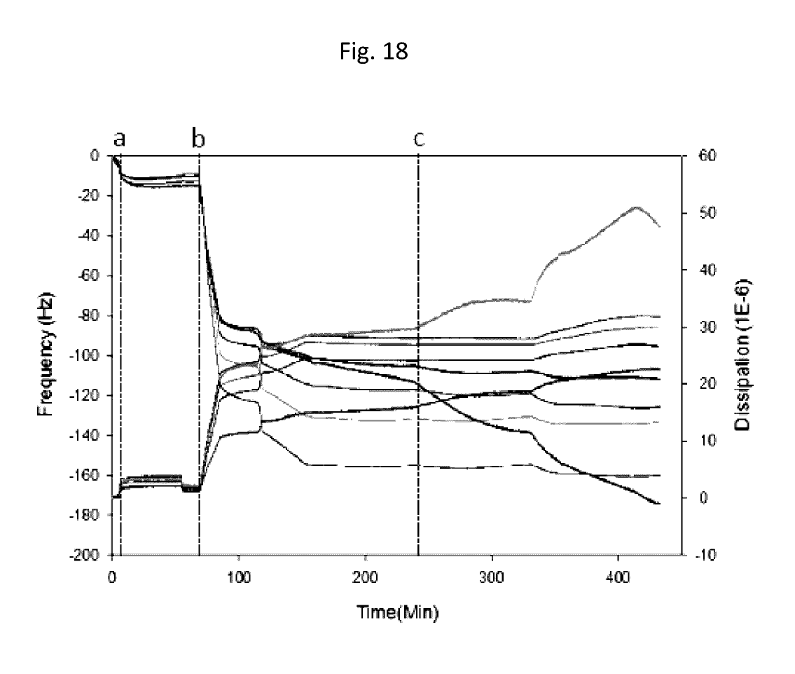

FIG. 18 illustrates a typical QCM-D response to grafting a M-CHY1 (SEQ ID NO: 3) anti-microbial peptide to a functionalized surface via a tether of PEG 7500, where frequency and dissipation are depicted on different axes versus time, at the 3rd, 5th, 7th, 9th and 11th overtones, and a-c represent major time stamps according to changes in material flow through the QCM-D; a=tether flow, b=peptide flow, c=bacteria flow. The interaction of M-CHY1 with a functionalized surface and PEG linking molecule is similar to that of C-CHY1 peptide attaching to the surface via PEG 7500 as monitored using QCM-D.

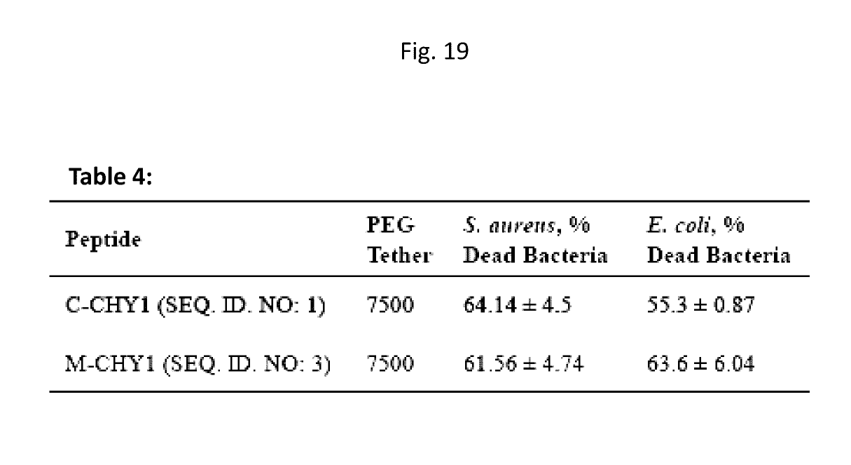

FIG. 19 includes Table 4, which is a comparison of the killing ability of PEG 7500-tethered C-CHY1 and M-CHY1 anti-microbial peptides tethered to a surface against Gram-negative E. coli and Gram-positive S. aureus bacteria. Table 4 demonstrates the percent dead bacteria (also called killing percent), Gram-positive S. aureus and Gram-negative E. coli, as a result of a 1 hour incubation with C-CHY1 and M-CHY1 covalently bound via PEG 7500 that had been incubated at room temperature (e.g. non-optimized system). Bacterial mortality was calculated by counting the number of live and number of dead bacteria in different areas on the QCM-D sensor and calculating the percent dead cells, for n=6 replicates and n=4 replicates for C-CHY1 and M-CHY1, respectively.

All patents, patent applications, and published references cited herein are hereby incorporated by reference in their entirety. It should be emphasized that the above-described embodiments of the present disclosure are merely possible examples of implementations, merely set forth for a clear understanding of the principles of the disclosure. Many variations and modifications may be made to the above-described embodiment(s) without departing substantially from the spirit and principles of the disclosure. It can be appreciated that several of the above-disclosed and other features and functions, or alternatives thereof, may be desirably combined into many other different systems or applications. All such modifications and variations are intended to be included herein within the scope of this disclosure, as fall within the scope of the appended claims.

What is claimed is:

1. A composition comprising a tether covalently attached to an anti-microbial peptide, the tether having sufficient length to permit the anti-microbial peptide to at least partially penetrate a membrane of a bacteria, upon contact of the anti-microbial peptide with the bacteria.

2. The composition of claim 1, wherein the anti-microbial peptide is derived from Chrysophsin-1, Chrysophsin-2, or Chrysophsin-3 by altering a terminal amino acid residue to covalently bind to the tether.

3. The composition of claim 1, wherein the anti-microbial peptide is a peptide consisting of the amino acid sequence of SEQ ID NO: 2, 3, 5, 6, 8 or 9.

4. The composition of claim 1, wherein the anti-microbial peptide is a peptide consisting of the amino acid sequence of SEQ ID NO: 2.

5. The composition of claim 1, wherein the anti-microbial peptide is a peptide consisting of the amino acid sequence of SEQ ID NO: 3.

6. The composition of claim 1, wherein the tether has a length between about 15 nm and about 75 nm.

7. The composition of claim 6, wherein the anti-microbial peptide is a peptide consisting of the amino acid sequence of SEQ ID NO: 1-9.

8. The composition of claim 1, wherein the tether comprises a hetero-bifunctionalized polymer, with a first end being terminated with a N-hydroxysuccinimide (NETS) and a second end being terminated with a maleimide group to covalently bind the second end of the tether to the anti-microbial peptide.

9. A medical device comprising:
a surface; and
an anti-microbial coating deposited on the surface, wherein the anti-microbial coating comprises: a tether covalently bound to the surface at a first end and covalently bound to an anti-microbial peptide at a second end, the tether having sufficient length to permit the anti-microbial peptide to at least partially penetrate a membrane of a bacteria, upon contact of the anti-microbial peptide with the bacteria.

10. The medical device of claim 9, wherein the anti-microbial peptide is derived from Chrysophsin-1, Chrysophsin-2, or Chrysophsin-3 by altering a terminal amino acid residue to covalently bind to the tether.

11. The medical device of claim 9, wherein the anti-microbial peptide is a peptide consisting of the amino acid sequence of SEQ ID NO: 2, 3, 5, 6, 8 or 9.

12. The medical device of claim 9, wherein the anti-microbial peptide is a peptide consisting of the amino acid sequence of SEQ ID NO: 2.

13. The medical device of claim 9, wherein the anti-microbial peptide is a peptide consisting of the amino acid sequence of SEQ ID NO: 3.

14. The medical device of claim 9, wherein the tether has a length between about 15 nm and about 75 nm.

15. The medical device of claim 14, wherein the anti-microbial peptide is a peptide consisting of the amino acid sequence of SEQ ID NO: 1-9.

16. The medical device of claim 9, wherein the tether comprises a hetero-bifunctionalized polymer, with a first end being terminated with a N-hydroxysuccinimide (NETS) to bind the first end of the tether to the surface and a second end being terminated with a maleimide group to covalently bind the second end of the tether to the anti-microbial peptide.

17. A method for preparing an anti-microbial medical device comprising:
applying a tether to a surface of a medical device to covalently attach the tether to the surface at a first end of the tether; and
binding an anti-microbial peptide to a second end of the tether, where the tether is selected to have characteristics allowing the tether to maintain or promote an anti-microbial activity of the anti-microbial peptide.

18. The method of claim 17 further comprising a step of functionalizing the surface to facilitate covalent binding of the tether to the surface.

19. The method of claim 17, wherein the anti-microbial peptide is a peptide consisting of the amino acid sequence of SEQ ID NO: 2, 3, 5, 6, 8 or 9.

20. The method of claim 17, wherein the tether has a length between about 15 nm and about 75 nm.


SEQUENCE LISTING (9 sequences)

SEQ ID NO: 1 - protein, 25 aa, Artificial Sequence
  Synthetic peptide
FFGWLIKGAIHAGKAIHGLIHRRRH

SEQ ID NO: 2 - protein, 26 aa, Artificial Sequence
  Synthetic peptide
CFFGWLIKGAIHAGKAIHGLIHRRRH

SEQ ID NO: 3 - protein, 26 aa, Artificial Sequence
  Synthetic peptide
MFFGWLIKGAIHAGKAIHGLIHRRRH

SEQ ID NO: 4 - protein, 25 aa, Artificial Sequence
  Synthetic peptide
FFGWLIRGAIHAGKAIHGLIHRRRH

SEQ ID NO: 5 - protein, 26 aa, Artificial Sequence
  Synthetic peptide
CFFGWLIRGAIHAGKAIHGLIHRRRH

SEQ ID NO: 6 - protein, 26 aa, Artificial Sequence
  Synthetic peptide
MFFGWLIRGAIHAGKAIHGLIHRRRH

SEQ ID NO: 7 - protein, 20 aa, Artificial Sequence
  Synthetic peptide
FIGLLISAGKAIHDLIRRRH

SEQ ID NO: 8 - protein, 21 aa, Artificial Sequence
  Synthetic peptide
CFIGLLISAGKAIHDLIRRRH

SEQ ID NO: 9 - protein, 21 aa, Artificial Sequence
  Synthetic peptide
MFIGLLISAGKAIHDLIRRRH